(19) United States
(12) Patent Application Publication
(10) Pub. No.: US 2016/0032397 A1
(43) Pub. Date: Feb. 4, 2016

(54) MAST CELL CANCER-ASSOCIATED GERM-LINE RISK MARKERS AND USES THEREOF

(71) Applicants: Malin MELIN, (US); Maja, Louise ARENDT, (US); THE BROAD INSTITUTE, INC., Cambridge, MA (US); TRUSTEES OF TUFTS COLLEGE, Boston, MA (US); ANIMAL HEALTH TRUST, Suffolk (GB)

(72) Inventor: Malin MELIN, Uppsala (SE)

(73) Assignees: The Broad Institute, Inc., Cambridge, MA (US); Trustees of Tufts College, Boston, MA (US); Animal Health Trust, Kentford, Newmarket (GB)

(21) Appl. No.: 14/774,836

(22) PCT Filed: Mar. 13, 2014
(86) PCT No.: PCT/US14/26385

§ 371 Date: Sep. 11, 2015

Related U.S. Application Data
(60) Provisional application No. 61/786,090, filed on Mar. 14, 2013.

(51) Int. Cl. C12Q 1/68

(52) U.S. Cl. CPC C12Q 1/6886; C12Q 2600/156; C12Q 2600/172

(57) ABSTRACT

Provided herein are methods and compositions for identifying subjects, including canine subjects, as having an elevated risk of developing cancer or having an undiagnosed cancer. These subjects are identified based on the presence of germ-line risk markers.

### CROSS-REFERENCE TO RELATED APPLICATIONS

This application claims the benefit of the filing date of U.S. Provisional Application No. 61/786,090, filed Mar. 14, 2013, the entire contents of which are incorporated by reference herein.

### FEDERALLY SPONSORED RESEARCH OR DEVELOPMENT

This invention was made with U.S. Government support under U54HG003067 awarded by the National Institutes of Health. The U.S. Government has certain rights in the invention. The research was also generously supported and funded by the Swedish government and Uppsala University.

### BACKGROUND OF INVENTION

Canine mast cell tumors (CMCTs) are one of the most common skin tumors in dogs with a major impact on canine health. Mast cells originate from the bone marrow and are normally found throughout the connective tissue of the body as normal components of the immune system. Mastocytosis is a term that covers a broad range of conditions characterized by the uncontrolled proliferation and infiltration of mast cells in tissues, and includes mastocytoma, mast cell cancer, and mast cell tumors. Common in these conditions is a high frequency of activating somatic mutations in the c-KIT oncogene [ref. 1,2]. An intriguing feature of the disease is its ability to spontaneously resolve despite having a mutation in an oncogene, as seen commonly in the juvenile condition[3]. Mast cell tumors in dogs share many phenotypic and molecular characteristics with human mastocytosis, including paraclinical and clinical manifestations and a high prevalence of activating c-KIT mutations [ref. 4-6]. Therefore, this disease in dogs provides a good naturally occurring comparative disease model for studying human mastocytosis. The nature of mast cell tumors in dogs is difficult to predict and accurate prognostication is challenging despite current classification schemes based on histopathology [Patnaik et al 1984, Kiupel et al. 2011]. Unclean surgical margins left after the surgical excision of a mast cell tumor can either relapse to regrow a new tumor or spontaneously regress [ref. 11].

### SUMMARY OF INVENTION

The invention is premised on the identification of germ-line risk markers (e.g., SNPs) that can be used singly or together (e.g., forming a haplotype) to predict elevated risk of mast cell cancer (MCC) in subjects, e.g., canine subjects. As described herein, a genome-wide association (GWAS) was performed in Golden Retrievers (GRs) and germ-line risk markers that correlate with canine MCC were identified. Accordingly, aspects of the invention provide methods for identifying subjects that are at elevated risk of developing MCC or subjects having otherwise undiagnosed MCC. Subjects are identified based on the presence of one or more germ-line risk markers shown to be associated with the presence of MCC, in accordance with the invention. Prognostic and theranostic methods utilizing one or more germ-line risk markers are also described herein.

Aspects of the invention relate to a method, comprising (a) analyzing genomic DNA from a canine subject for the presence of a single nucleotide polymorphism (SNP) selected from:
i) one or more chromosome 5 SNPs,
ii) a chromosome 8 SNP TIGRP2P118921,
iii) one or more chromosome 14 SNPs, and
iv) one or more chromosome 20 SNPs; and
   (b) identifying a canine subject having the SNP as a subject at elevated risk of developing a mast cell cancer or having an undiagnosed mast cell cancer. In some embodiments, the SNP is selected from one or more chromosome 14 SNPs and one or more chromosome 20 SNPs.

In some embodiments, the SNP is selected from one or more chromosome 14 SNPs. In some embodiments, the SNP is selected from one or more chromosome 14 SNPs BICF2G630521558, BICF2G630521606, BICF2G630521619, BICF2G630521572, and BICF2P867665. In some embodiments, the SNP is BICF2P867665. In some embodiments, the canine subject is of American descent.

In some embodiments, the SNP is selected from one or more chromosome 20 SNPs. In some embodiments, the SNP is selected from one or more chromosome 20 SNPs BICF2S22934685, BICF2P1444805, BICF2P299292, BICF2P301921, and BICF2P623297. In some embodiments, the SNP is BICF2P301921. In some embodiments, the canine subject is of European descent.

In some embodiments, the SNP is selected from one or more chromosome 20 SNPs BICF2P304809, BICF2P1310301, BICF2P1310305, BICF2P1231294, and BICF2P1185290. In some embodiments, the SNP is BICF2P1185290. In some embodiments, the canine subject is of European descent or American descent.

In some embodiments, the SNP is two or more SNPs. In some embodiments, the SNP is three or more SNPs.

Other aspects of the invention relate to a method, comprising (a) analyzing genomic DNA from a canine subject for the presence of a risk haplotype selected from:
(i) a risk haplotype having chromosome coordinates Chr5:8.42-10.73 Mb,
(ii) a risk haplotype having chromosome coordinates Chr14:14.64-14.76 Mb,
(iii) a risk haplotype having chromosome coordinates Chr20:41.51-42.12 Mb,
(iv) a risk haplotype having chromosome coordinates Chr20:41.70-42.59 Mb, and
(v) a risk haplotype having chromosome coordinates Chr20:47.06-49.70 Mb; and
   (b) identifying a canine subject having the risk haplotype as a subject at elevated risk of developing a mast cell cancer or having an undiagnosed mast cell cancer.

In some embodiments, the presence of the risk haplotype is detected by analyzing the genomic DNA for the presence of a SNP is selected from:

(a) Chr5:8.42-10.73 Mb SNPs BICF2P807873, BICF2P778319, BICF2P547394, BICF2P1347656, BICF2S2331073, BICF2S23025903, and BICF2S23519930,

(b) Chr14:14.64-14.76 Mb SNPs BICF2G630521558, BICF2G630521572, BICF2G630521606, BICF2G630521619, BICF2P867665, TIGRP2P186605, BICF2G630521678, BICF2G630521681, and BICF2G630521696,

(c) Chr20:41.51-42.12 Mb SNPs BICF2P453555, BICF2P372450, BICF2P271393, BICF2S22934685, BICF2S2295117,

(d) Chr20:41.70-42.59 Mb SNPs BICF2P453555, BICF2P372450, BICF2P271393, BICF2S22934685, BICF2S2295117, and

(e) Chr20:47.06-49.70 Mb SNPs BICF2P327134, BICF2P854185, BICF2P304809, BICF2P1310301, BICF2P1310305, BICF2P1231294, BICF2P541405, BICF2P112281, BICF2P1185290, and BICF2P1241961. In some embodiments, the risk haplotype is selected from the risk haplotype having chromosome coordinates Chr14:14.64-14.76 Mb, the risk haplotype having chromosome coordinates Chr20:41.51-42.12 Mb, the risk haplotype having chromosome coordinates Chr20:41.70-42.59 Mb, and the risk haplotype having chromosome coordinates Chr20:47.06-49.70 Mb.

In some embodiments, the risk haplotype is the risk haplotype having chromosome coordinates Chr14:14.64-14.76 Mb. In some embodiments, the canine subject is of American descent.

In some embodiments, the risk haplotype is the risk haplotype having chromosome coordinates Chr20:41.51-42.12 Mb. In some embodiments, the canine subject is of American or European descent.

In some embodiments, the risk haplotype is the risk haplotype having chromosome coordinates Chr20:41.70-42.59 Mb or the risk haplotype having chromosome coordinates Chr20:47.06-49.70 Mb. In some embodiments, the risk haplotype is the risk haplotype having chromosome coordinates Chr20:47.06-49.70 Mb. In some embodiments, the canine subject is of European descent.

In some embodiments, the SNP is two or more SNPs. In some embodiments, the SNP is three or more SNPs. In some embodiments, the SNP is a group of SNPs selected from (a) to (e):

(a) Chr5:8.42-10.73 Mb SNPs BICF2P807873, BICF2P778319, BICF2P547394, BICF2P1347656, BICF2S2331073, BICF2S23025903, and BICF2S23519930,

(b) Chr14:14.64-14.76 Mb SNPs BICF2G630521558, BICF2G630521572, BICF2G630521606, BICF2G630521619, BICF2P867665, TIGRP2P186605, BICF2G630521678, BICF2G630521681, and BICF2G630521696,

(c) Chr20:41.51-42.12 Mb SNPs BICF2P453555, BICF2P372450, BICF2P271393, BICF2S22934685, BICF2S2295117,

(d) Chr20:41.70-42.59 Mb SNPs BICF2P453555, BICF2P372450, BICF2P271393, BICF2S22934685, BICF2S2295117, and

(e) Chr20:47.06-49.70 Mb SNPs BICF2P327134, BICF2P854185, BICF2P304809, BICF2P1310301, BICF2P1310305, BICF2P1231294, BICF2P541405, BICF2P112281, BICF2P1185290, and BICF2P1241961.

In some embodiments, the risk haplotype is two or more risk haplotypes. In some embodiments, the risk haplotype is three or more risk haplotypes.

In another aspect, the invention relates to a method, comprising (a) analyzing genomic DNA from a canine subject for the presence of a mutation in a gene selected from:

(i) one or more genes located within a risk haplotype having chromosome coordinates Chr5:8.42-10.73 Mb,

(ii) one or more genes within 500 Kb of TIGRP2P118921 on chromosome 8,

(iii) one or more genes located within a risk haplotype having chromosome coordinates Chr14:14.64-14.76 Mb,

(iv) one or more genes located within a risk haplotype having chromosome coordinates Chr20:41.51-42.12 Mb,

(v) one or more genes located within a risk haplotype having chromosome coordinates Chr20:41.70-42.59 Mb, and

(vi) one or more genes located within a risk haplotype having chromosome coordinates Chr20:47.06-49.70 Mb, and

(b) identifying a canine subject having the mutation as a subject at elevated risk of developing a mast cell cancer or having an undiagnosed mast cell cancer.

In some embodiments, the gene is selected from one or more genes located within a risk haplotype having chromosome coordinates Chr14:14.64-14.76 Mb. In some embodiments, the gene is selected from SPAM1, HYAL4, and HYALP1. In some embodiments, the canine subject is of American descent.

In some embodiments, the gene is selected from one or more genes located within a risk haplotype having chromosome coordinates Chr20:41.51-42.12 Mb or one or more genes located within a risk haplotype having chromosome coordinates Chr20:47.06-49.70 Mb. In some embodiments, the gene is selected from one or more genes located within a risk haplotype having chromosome coordinates Chr20:47.06-49.70 Mb. In some embodiments, the canine subject is of European descent.

In some embodiments, the gene is selected from one or more genes located within a risk haplotype having chromosome coordinates Chr20:41.51-42.12 Mb. In some embodiments, the gene is selected from DOCK3, ENSCAFG00000010275, MAPKAPK3, CISH, HEMK1, C3orf18, CACNA2D2, TMEM115, NPRL2, ZMYND10, RASSF1, TUSC2, HYAL2, HYAL1, HYAL3, C3orf45, ENSCAFG00000010719, GNAI2_CANFA, and ENSCAFG00000010754. In some embodiments, the canine subject is of American or European descent.

In some embodiments, the gene is selected from MAPKAPK3, CISH, HEMK1, C3orf18, CACNA2D2, TMEM115, CYB561D2, NPRL2, ZMYND10, RASSF1, TUSC2, HYAL2, HYAL1, HYAL3, C3oef45, GNAI2, ENSCAFG00000010719, and ENSCAFG00000010754.

In some embodiments, the gene is GNAI2. In some embodiments, the gene is selected from HYAL1, HYAL2, HYAL3, SPAM1, HYAL4, and HYALP1. In some embodiments, the gene is selected from HYAL1, HYAL2, HYAL3, SPAM1, HYAL4, HYALP1, and TMEM229A.

In some embodiments, the mutation is two or more mutations. In some embodiments, the mutation is three or more mutations. In some embodiments, the gene is two or more genes. In some embodiments, the gene is three or more genes.

In some embodiments of any of the methods provided herein, the genomic DNA is obtained from a bodily fluid or tissue sample of the subject. In some embodiments, the genomic DNA is obtained from a blood or saliva sample of the subject.

In some embodiments of any of the methods provided herein, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay.

In some embodiments of any of the methods provided herein, the mast cell cancer is a mast cell cancer located in the skin of the subject.

In some embodiments of any of the methods provided herein, the canine subject is a descendent of a Golden Retriever. In some embodiments, the canine subject is a Golden Retriever.

Other aspects of the invention relate to a method, comprising (a) analyzing genomic DNA in a sample from a subject for presence of a mutation in a gene selected from
(i) one or more genes located within a risk haplotype having chromosome coordinates Chr5:8.42-10.73 Mb, or an orthologue of such a gene,
(ii) one or more genes within 500 Kb of TIGRP2P118921 on chromosome 8,
(iii) one or more genes located within a risk haplotype having chromosome coordinates Chr14:14.64-14.76 Mb, or an orthologue of such a gene,
(iv) one or more genes located within a risk haplotype having chromosome coordinates Chr20:41.51-42.12 Mb, or an orthologue of such a gene,
(v) one or more genes located within a risk haplotype having chromosome coordinates Chr20:41.70-42.59 Mb, or an orthologue of such a gene, and
(vi) one or more genes located within a risk haplotype having chromosome coordinates Chr20:47.06-49.70 Mb or an orthologue of such a gene; and
   (b) identifying a subject having the mutation as a subject at elevated risk of developing a mast cell cancer or having an undiagnosed mast cell cancer.

In some embodiments, the subject is a human subject. In some embodiments, the subject is a canine subject.

In some embodiments, the genomic DNA is obtained from a bodily fluid or tissue sample of the subject. In some embodiments, the genomic DNA is obtained from a blood or saliva sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay. In some embodiments, the mast cell cancer is a mast cell cancer located in the skin of the subject.

In some embodiments, the gene is two or more genes. In some embodiments, the gene is three or more genes. In some embodiments, the mutation is two or more mutations. In some embodiments, the mutation is three or more mutations.

### BRIEF DESCRIPTION OF DRAWINGS

FIG. 1 is a multi-dimensional scaling plot displaying the first two dimensions, C1 and C2, showing (1) the overall genetic similarity between the individuals in the study and (2) that American and European dogs form two clusters according to continent. The majority of American dogs cluster on the right side of the plot while the majority of the European dogs cluster of the left side of the plot.

FIG. 2 is a series of quantile-quantile plots (left) and Manhattan plots (right) showing the GWAS results for the GR cohort. The nominal significance levels of the quantile-quantile (QQ) plots are indicated by the dashed lines, based on where the observed values fall outside the confidence interval for expected values. The Manhattan plots display −log p values with cut-offs based on QQ plots. (A) In American GRs a major locus is seen on chromosome 14, with weaker nominally significant SNPs on two additional chromosomes. (B) In European GRs the strongest association is seen on chromosome 20, with weaker signals on 9 additional chromosomes. There is no overlap in loci detected in the European and American cohorts. (C) A combined analysis results in a strengthened association on chromosome 20.

FIG. 3 is a series of graphs depicting the regional association results for chromosome 14 in the American cohort. (A) Association plot and (B) minor allele frequency plot for chromosome 14. (C) Candidate region with dots shaded according to pair-wise linkage disequilibrium (LD) with the top SNP. The degree of shading in the objects corresponds to LD with the top SNP, with 5 different grades of shading from lightest to darkest indicating: <0.2, 0.2-0.4, 0.4-0.6, 0.6-0.8, and 0.8-1.0. (D) The top haplotype spans a region containing three genes: SPAM1, HYAL4 and HYALP1. Horizontal black arrows indicate direction of transcription and the vertical black arrow indicate the top SNP position.

FIG. 4 is a series of graphs showing the European GWAS results for chromosome 20. (A) Association plot and (B) minor allele frequency plot for chromosome 20. Note the reduction in minor allele frequencies near the top associations. (C) Candidate region with dots shaded according to pair-wise LD with the top SNP in the 49 Mb locus. The degree of shading in the objects corresponds to LD with the top SNP, with 5 different grades of shading from lightest to darkest indicating: <0.2, 0.2-0.4, 0.4-0.6, 0.6-0.8, and 0.8-1.0. (D) Candidate region with dots shaded according to pair-wise LD with the top SNP in the 42 Mb locus. The degree of shading in the objects corresponds to LD with the top SNP, with 5 different grades of shading from lightest to darkest indicating: <0.2, 0.2-0.4, 0.4-0.6, 0.6-0.8, and 0.8-1.0. (E) The genes located within the top haplotype are marked with black bars. The black arrow indicates the position of the top SNP.

FIG. 5 is a series of graphs depicting the association results for chromosome 20 in the full GR cohort. (A) Association plot and (B) minor allele frequency plot for chromosome 20. (C) Candidate region with dots shaded according to pair-wise LD with the top SNP. The degree of shading in the objects corresponds to LD with the top SNP, with 5 different grades of shading from lightest to darkest indicating: <0.2, 0.2-0.4, 0.4-0.6, 0.6-0.8, and 0.8-1.0. (D) The genes located within the top haplotype are marked with black bars. The arrow indicates the position of the top SNP.

FIG. 6 is a series of bar graphs depicting SNP risk genotype frequencies and risk haplotype frequencies in the cohorts. Black=homozygous risk, grey=heterozygotes and white=homozygous protective. (A) Chr14:14.7 Mb, (B) Chr20:42.5 Mb, (C) Chr20:48.6 Mb, (D) Chr:2041.9 Mb).

FIG. 7 is a series of two multi-dimensional scaling plots showing a relatively uniform distribution within continental clusters. (A) American GR cases and controls (B) European cases and controls.

FIG. 8 is a QQ plot of the full cohort after removal of region 27.5 Mb—50.5 Mb on chromosome 20. The genomic inflation factor is 0.97.

FIG. 9 is a gel image showing PCR products formed using a splice specific 5′ primer traversing across exon 2 and 4 hence excluding exon 3. Only individuals with the T risk genotype produce the alternative splice product.

FIG. 10. is an illustration of the splice specific primer design. The 5′ primer expands over exon 2 and 4 and thereby skips exon 3. A PCR product will only form if the alternative splice form, which splices out exon 3, is present in the cDNA template.

### DETAILED DESCRIPTION OF INVENTION

Mast cell cancer (MCC) occurs commonly in canines and has a major impact on canine health. MCC also occurs in other animals, including humans and felines. Modern dog breeds have been created by extensive selection for certain phenotypic characteristics. As a side effect, there has been enrichment of unwelcome traits, such as increased risk of developing a disease or condition.

Aspects of the invention relate to germ-line risk markers (such as single nucleotide polymorphisms (SNPs), risk haplotypes, and mutations in genes) and various methods of use and/or detection thereof. The invention is premised, in part, on the results of a case-control GWAS of 252 GRs performed to identify germ-line risk markers associated with MCC. The study is described herein. Briefly, SNPs were identified that correlate with the presence of MCC in American and European GRs. Significant SNPs were identified on chromosomes 5, 8, 14, and 20. These SNPs are listed in Table 1A and in Table 1B. Additionally, risk haplotypes consisting of chromosomal regions on chromosomes 5, 14 and 20 were identified that significantly correlated with MCC in the GRs (Chr5:8.42-10.73 Mb, Chr14:14.64-14.76 Mb, Chr20:41.51-42.12 Mb, Chr20:41.70-42.59 Mb, and Chr20:47.06-49.70 Mb).

Accordingly, aspects of the invention provide methods that involve detecting one or more of the identified germ-line risk markers in a subject, e.g., a canine subject, in order to (a) identify a subject at elevated risk of developing a MCC, or (b) identify a subject having a MCC that is as yet undiagnosed. The methods can be used for prognostic purposes and for diagnostic purposes. Identifying canine subjects having an elevated risk of developing a MCC is useful in a number of applications. For example, canine subjects identified as at elevated risk may be excluded from a breeding program and/or conversely canine subjects that do not carry the germ-line risk markers may be included in a breeding program. As another example, canine subjects identified as at elevated risk may be monitored, including monitored more regularly, for the appearance of MCC and/or may be treated prophylactically (e.g., prior to the development of the tumor) or therapeutically. Canine subjects carrying one or more of the germ-line risk markers may also be used to further study the progression of MCC and optionally to study the efficacy of various treatments.

In addition, in view of the clinical and histological similarity between canine MCC with human MCC [see, e.g., ref. 4-6], the germ-line risk markers identified in accordance with the invention may also be risk markers and/or mediators of cancer occurrence and progression in human MCC as well. Accordingly, the invention provides diagnostic and prognostic methods for use in canine subjects, animals more generally, and human subjects, as well as animal models of human disease and treatment, as well as others.

Additionally, two of the most strongly MCC-associated chromosomal regions (Chr14:14.64-14.76 Mb, Chr20:41.51-42.12 Mb, and Chr20:41.70-42.59 Mb) identified in the GWAS study were found to contain hyaluronidase enzyme genes. For example, one of the most significant SNPs on chromosome 14 (BICF2P867665) was found to be located in the second intron of hyaluronidase gene HYALP1. Hyaluronidase enzymes degrade the glucosaminoglycan hyaluronic acid (HA), which is a major component of the extracellular matrix and cellular microenvironment. The aforementioned chromosomal regions contain genes involved in HA degradation. Without wishing to be bound by theory, this finding suggests that the HA pathway may be involved in canine MCC predisposition or progression. The biological function of HA depends on its molecular mass. Again, without wishing to be bound by theory, up-regulation of hyaluronidase activity may lead to expansion of the mast cell population by converting high molecular weight HA to low molecular weight HA [ref. 27]. Hyaluronidase mutations, such as those identified in the GR cohort, may change the HA balance, which in turn may modify the extracellular environment of to create a favorable tumor microenvironment.

Accordingly, additional aspects of the invention provide methods that involve detecting one or more mutations in one or more hyaluronidase genes in a subject, e.g., a canine subject, in order to (a) identify a subject at elevated risk of developing a MCC or (b) identify a subject having a MCC that is present but undiagnosed. Other aspects of the invention relate to treatment of MCC in a subject through blockade of HA signaling (e.g., by degrading HA, by degrading a receptor for HA, such as CD44, or by blocking the interaction of HA and the receptor for HA, e.g., CD44). In some embodiments, treatment comprises administering a CD44 inhibitor and/or an HA inhibitor to a subject with MCC.

### Elevated Risk of Developing Mast Cell Cancer

The germ-line risk markers of the invention can be used to identify subjects at elevated risk of developing a mast cell cancer (MCC). An elevated risk means a lifetime risk of developing such a cancer that is higher than the risk of developing the same cancer in (a) a population that is unselected for the presence or absence of the germ-line risk marker (i.e., the general population) or (b) a population that does not carry the germ-line risk marker.

### Mast Cell Cancer and Diagnostic/Prognostic Methods

Aspects of the invention include various methods, such as prognostic and diagnostic methods, related to mast cell cancer (MCC). MCC occurs when mast cells proliferate uncontrollably and/or invade tissues in the body. In canines, MCC tumors (also referred to as mast cell tumors, MCTs) are often found in the skin and may present as a wart-like nodule, a soft subcutaneous lump, or an ulcerated skin mass [see, e.g., Moore, Anthony S. (2005). “Cutaneous Mast Cell Tumors in Dogs”. Proceedings of the 30th World Congress of the World Small Animal Veterinary Association and “Cutaneous Mast Cell Tumors”. The Merck Veterinary Manual. (2006)]. However, it is to be appreciated that MCC can be located in other tissues besides the skin, including, for example, within the gastrointestinal tract or a lymph node. The invention provides methods for detecting germ-line risk markers regardless of the location of the cancer.

Currently available methods for diagnosis of MCC typically involve a needle aspiration biopsy at the site of a suspected tumor. Mast cells are identified by their granules, which stain blue to dark purple with a Romanowsky stain. Further or alternative diagnosis may involve a surgical biopsy, which can be used to determine the grade of the cancer. X-rays, ultrasound, or lymph node, bone marrow, or organ biopsies may also be used to stage the cancer. MCCs can be staged according to the WHO criteria [see, e.g., Morrison, Wallace B. (1998). Cancer in Dogs and Cats (1st ed.). Williams and Wilkins] which includes:

Stage I—a single skin tumor with no spread to lymph nodes

Stage II—a single skin tumor with spread to lymph nodes in the surrounding area

Stage III—multiple skin tumors or a large tumor invading deep to the skin with or without lymph node involvement, and

Stage IV—a tumor with metastasis to the spleen, liver, bone marrow, or with the presence of mast cells in the blood.

Alternatively, or additionally, MCTs may be graded using a grading system, which includes:

Grade I—well differentiated and mature cells with a low potential for metastasis,

Grade II—intermediately differentiated cells with potential for local invasion and moderate metastatic behavior, and

Grade III—undifferentiated, immature cells with a high potential for metastasis.

In addition, activating c-KIT mutations and/or levels of c-KIT are also used to diagnose MCC [ref. 1,2]. For example, PCR may be used to detect activating mutations in the c-KIT gene and/or immunohistochemical staining of a biopsy may be used to detect elevated c-KIT levels. Detection of c-KIT mutations and/or levels may be used to identify subjects to be treated with tyrosine kinase inhibitors (e.g., Toceranib, Masitinib).

Thus, in some embodiments, the prognostic or diagnostic methods of the invention may further comprise performing a diagnostic assay known in the art for identification of a MCC (e.g., fine needle aspirate based cytology, biopsy, X-ray, detection of c-KIT mutations, detection of c-KIT levels and/or ultrasound).

Germ-Line Risk Markers

Aspects of the invention relate to germ-line risk markers and use and detection thereof in various methods. In general terms, a germ-line marker is a mutation in the genome of a subject that can be passed on to the offspring of the subject. Germ-line markers may or may not be risk markers. Germ-line markers are generally found in the majority, if not all, of the cells in a subject. Germ-line markers are generally inherited from one or both parents of the subject (was present in the germ cells of one or both parents). Germ-line markers as used herein also include de novo germ-line mutations, which are spontaneous mutations that occur at single-cell stage level during development. This is distinct from a somatic marker, which is a mutation in the genome of a subject that occurs after the single-cell stage during development. Somatic mutations are considered to be spontaneous mutations. Somatic mutations generally originate in a single cell or subset of cells in the subject.

A germ-line risk marker as described herein includes a SNP, a risk haplotype, or a mutation in a gene. Further discussion of each type of germ-line risk marker is described herein. It is to be understood that a germ-line risk marker may also indicate or predict the presence of a somatic mutation in a genomic location in close proximity to the germ-line risk marker, as germ-line risk marks may correlate with a higher risk of secondary somatic mutations.

As used herein, a mutation is one or more changes in the nucleotide sequence of the genome of the subject. The terms mutation, alteration, variation, and polymorphism are used interchangeably herein. As used herein, mutations include, but are not limited to, point mutations, insertions, deletions, rearrangements, inversions and duplications. Mutations also include, but are not limited to, silent mutations, missense mutations, and nonsense mutations.

### Single Nucleotide Polymorphisms (SNPs)

In some embodiments, a germ-line risk marker is a single nucleotide polymorphism (SNP). A SNP is a mutation that occurs at a single nucleotide location on a chromosome. The nucleotide located at that position may differ between individuals in a population and/or paired chromosomes in an individual. In some embodiments, a germ-line risk marker is a SNP selected from Table 1A. In some embodiments, a germ-line risk marker is a SNP selected from Table 1B. Table 1A and Table 1B provide the non-risk and risk nucleotide identity for each SNP. The “REF” column of Table 1A and Table 1B refers to the nucleotide identity present in the Boxer reference genome. The risk nucleotide is the nucleotide identity that is associated with elevated risk of developing a MCC or having an undiagnosed MCC. The position (i.e. the chromosome coordinates) and SNP ID for each SNP in Table 1A and Table 1B are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The first base pair in each chromosome is labeled 0 and the position of the SNP is then the number of base pairs from the first base pair (for example, the SNP chr20:41488878 is located 41488878 base pairs from the first base pair of chromosome 20).

TABLE 1A
List of SNPs associated with elevated risk of mast cell cancer

| | | | NUCLEOTIDE | | | | |
|---|---|---|---|---|---|---|---|
| | | | IDENTITY | | | Frequency | Frequency |
| | CHROMO- | | (NON - | SIGNIFI- | | risk allele | risk allele |
| SNP ID | SOME | POSITION | RISK/RISK) | CANCE | Ref | cases | controls |
| BICF2P807873 | 5 | 8428475 | A/G | 3.07E−04 | G | 0.892 | 0.8333 |
| BICF2P778319 | 5 | 8431406 | T/C | 3.07E−04 | C | 0.892 | 0.8291 |
| BICF2P547394 | 5 | 8487193 | A/G | 3.07E−04 | G | 0.892 | 0.8376 |
| BICF2P1347656 | 5 | 9397630 | A/T | 3.07E−04 | T | 0.892 | 0.8376 |
| BICF2P1471782 | 5 | 10511987 | C/G | 1.74E−04 | C | 0.812 | 0.6966 |
| BICF2P1198876 | 5 | 10565740 | G/A | 1.04E−04 | G | 0.78 | 0.641 |
| BICF2S2331073 | 5 | 10667930 | T/C | 1.94E−04 | T | 0.772 | 0.6325 |
| BICF2S23025903 | 5 | 10709446 | A/G | 1.94E−04 | G | 0.772 | 0.6325 |
| BICF2S23519930 | 5 | 10728844 | G/A | 4.47E−05 | A | 0.8 | 0.6496 |
| BICF2P27872 | 5 | 11222952 | C/T | 2.16E−04 | T | 0.632 | 0.5128 |
| BICF2P27877 | 5 | 11225752 | T/C | 3.19E−04 | C | 0.624 | 0.5043 |
| BICF2P1035987 | 5 | 11380134 | G/A | 5.70E−04 | A | 0.72 | 0.5513 |
| TIGRP2P118921 | 8 | 66741586 | C/T | 4.09E−05 | C | 0.828 | 0.7565 |
| BICF2G630521558 | 14 | 14644897 | T/C | 1.24E−06 | C | 0.568 | 0.3932 |
| BICF2G630521572 | 14 | 14670361 | C/T | 3.41E−06 | T | 0.384 | 0.2051 |
| BICF2G630521606 | 14 | 14682089 | C/T | 2.47E−06 | T | 0.568 | 0.4017 |
| BICF2G630521619 | 14 | 14685543 | T/C | 1.24E−06 | C | 0.572 | 0.4017 |
| BICF2P867665 | 14 | 14714009 | T/G | 5.53E−07 | T | 0.56 | 0.3803 |
| TIGRP2P186605 | 14 | 14727905 | A/G | 5.48E−06 | G | 0.38 | 0.2009 |
| BICF2G630521678 | 14 | 14740313 | G/A | 5.48E−06 | G | 0.38 | 0.2051 |
| BICF2G630521681 | 14 | 14743663 | T/C | 5.48E−06 | T | 0.38 | 0.2051 |
| BICF2G630521696 | 14 | 14756089 | A/G | 3.41E−06 | A | 0.384 | 0.2051 |
| BICF2P626537 | 14 | 15009328 | G/A | 2.29E−04 | G | 0.268 | 0.1282 |
| BICF2G630521963 | 14 | 15089124 | A/G | 1.75E−04 | A | 0.272 | 0.1282 |
| BICF2G630522103 | 14 | 15197824 | T/C | 1.75E−04 | C | 0.268 | 0.1282 |
| BICF2G630522165 | 14 | 15379606 | A/C | 3.00E−05 | C | 0.588 | 0.4402 |
| BICF2P1423766 | 20 | 34594689 | T/C | 1.95E−04 | T | 0.648 | 0.5043 |
| BICF2P652049 | 20 | 34619934 | G/A | 1.95E−04 | G | 0.648 | 0.5 |
| BICF2P995880 | 20 | 34755165 | C/G | 1.59E−04 | G | 0.652 | 0.5085 |
| BICF2P1320326 | 20 | 34856730 | A/C | 1.10E−04 | C | 0.652 | 0.5043 |
| BICF2P1425181 | 20 | 34934336 | T/C | 2.78E−04 | C | 0.648 | 0.5085 |
| BICF2S23333987 | 20 | 36006050 | T/A | 5.41E−05 | T | 0.68 | 0.4783 |
| G1102F25S86 | 20 | 36081820 | C/T | 3.70E−04 | C | 0.536 | 0.3718 |
| BICF2S2309267 | 20 | 36310170 | G/A | 8.08E−05 | G | 0.688 | 0.4872 |
| BICF2S23432636 | 20 | 36319043 | C/A | 2.08E−04 | C | 0.572 | 0.3718 |
| BICF2S2343757 | 20 | 36431095 | C/T | 1.73E−04 | C | 0.572 | 0.3718 |
| BICF2S2355724 | 20 | 36435937 | T/G | 3.61E−05 | T | 0.524 | 0.3248 |
| BICF2P1078264 | 20 | 36638018 | T/C | 5.74E−05 | T | 0.524 | 0.3291 |
| BICF2P1110958 | 20 | 37772947 | G/A | 1.00E−04 | A | 0.576 | 0.3932 |
| BICF2P247805 | 20 | 38507160 | T/C | 4.34E−05 | T | 0.628 | 0.4615 |
| BICF2P1294383 | 20 | 38524299 | G/A | 7.06E−05 | G | 0.628 | 0.4658 |
| TIGRP2P274298 | 20 | 38744377 | A/G | 6.53E−05 | G | 0.64 | 0.4701 |
| BICF2S23549218 | 20 | 38864849 | C/G | 1.07E−05 | G | 0.708 | 0.5342 |
| BICF2P272829 | 20 | 39056905 | G/A | 1.56E−04 | A | 0.768 | 0.6239 |
| BICF2P1015829 | 20 | 39117538 | G/C | 2.97E−04 | C | 0.768 | 0.6207 |
| BICF2P948355 | 20 | 39134215 | T/C | 2.97E−04 | C | 0.768 | 0.6282 |
| BICF2S23620989 | 20 | 39138554 | C/T | 2.97E−04 | T | 0.768 | 0.6282 |
| BICF2P1081825 | 20 | 39156399 | G/C | 1.65E−05 | C | 0.612 | 0.4231 |
| BICF2S23418753 | 20 | 39230593 | T/C | 5.44E−05 | T | 0.624 | 0.453 |
| TIGRP2P274409 | 20 | 39317496 | A/C | 1.28E−04 | A | 0.6 | 0.4231 |
| BICF2S23344904 | 20 | 39351635 | T/C | 4.04E−05 | C | 0.608 | 0.4217 |
| BICF2S23749844 | 20 | 39354310 | A/G | 4.04E−05 | G | 0.608 | 0.4274 |
| BICF2P1242966 | 20 | 39365169 | T/C | 4.24E−06 | C | 0.652 | 0.4744 |
| BICF2S23450151 | 20 | 39397583 | C/A | 6.00E−06 | A | 0.652 | 0.4829 |
| BICF2P88083 | 20 | 39777883 | A/G | 1.08E−04 | G | 0.688 | 0.5043 |
| BICF2S23447001 | 20 | 39787259 | A/G | 2.89E−04 | A | 0.684 | 0.5085 |
| BICF2S23448192 | 20 | 39794609 | A/G | 2.89E−04 | A | 0.684 | 0.5085 |
| BICF2P619863 | 20 | 39803010 | C/T | 5.66E−05 | T | 0.696 | 0.5085 |
| BICF2P560295 | 20 | 39815670 | C/T | 5.66E−05 | T | 0.696 | 0.5085 |
| BICF2S2368248 | 20 | 40270272 | A/G | 2.31E−04 | G | 0.664 | 0.5171 |
| BICF2P279450 | 20 | 40635275 | T/G | 1.82E−04 | G | 0.692 | 0.5299 |
| TIGRP2P274855 | 20 | 41180269 | A/G | 4.76E−06 | G | 0.756 | 0.594 |
| BICF2P1314689 | 20 | 41215117 | C/A | 2.92E−05 | A | 0.712 | 0.5641 |
| BICF2P914653 | 20 | 41217592 | C/T | 2.92E−05 | T | 0.712 | 0.5641 |
| BICF2P408113 | 20 | 41229381 | T/G | 2.92E−05 | G | 0.712 | 0.5641 |
| BICF2P116133 | 20 | 41241178 | A/G | 2.92E−05 | G | 0.712 | 0.5603 |
| TIGRP2P274858 | 20 | 41271157 | T/G | 1.27E−05 | G | 0.7621 | 0.615 |
| BICF2P471574 | 20 | 41291981 | T/C | 2.92E−05 | C | 0.712 | 0.5603 |
| BICF2S23114565 | 20 | 41304489 | G/A | 2.92E−05 | A | 0.712 | 0.5641 |
| BICF2P509577 | 20 | 41310875 | A/C | 2.92E−05 | C | 0.712 | 0.5641 |
| BICF2P735611 | 20 | 41327714 | A/G | 2.92E−05 | G | 0.712 | 0.5641 |
| BICF2P1224909 | 20 | 41337123 | A/G | 2.92E−05 | G | 0.712 | 0.5641 |
| BICF2P413074 | 20 | 41345712 | G/A | 2.92E−05 | A | 0.712 | 0.5641 |
| BICF2P626859 | 20 | 41365616 | G/A | 2.92E−05 | A | 0.712 | 0.5641 |
| BICF2P968727 | 20 | 41387018 | C/T | 2.92E−05 | T | 0.712 | 0.5641 |
| BICF2P1139808 | 20 | 41395277 | C/T | 2.92E−05 | T | 0.712 | 0.5641 |
| BICF2P1342476 | 20 | 41411067 | G/A | 2.92E−05 | A | 0.712 | 0.5641 |
| BICF2P769104 | 20 | 41422308 | C/T | 2.92E−05 | T | 0.712 | 0.5641 |
| BICF2P648601 | 20 | 41424761 | G/A | 2.92E−05 | A | 0.712 | 0.5641 |
| BICF2P789266 | 20 | 41454760 | G/A | 2.92E−05 | A | 0.712 | 0.5641 |
| BICF2P549 | 20 | 41466952 | A/G | 1.87E−05 | G | 0.712 | 0.5603 |
| BICF2P257870 | 20 | 41488878 | G/A | 2.92E−05 | A | 0.712 | 0.5641 |
| BICF2S23351441 | 20 | 41493229 | C/A | 2.92E−05 | A | 0.712 | 0.5641 |
| BICF2P327134 | 20 | 41516957 | C/A | 1.13E−06 | A | 0.652 | 0.4957 |
| BICF2P20683 | 20 | 41576457 | A/G | 1.87E−05 | G | 0.712 | 0.5565 |
| BICF2P360884 | 20 | 41586182 | C/T | 2.92E−05 | T | 0.712 | 0.5641 |
| BICF2P1163972 | 20 | 41618769 | A/C | 2.92E−05 | C | 0.712 | 0.5641 |
| BICF2P983977 | 20 | 41642791 | C/T | 3.58E−05 | T | 0.712 | 0.5647 |
| BICF2P687775 | 20 | 41662902 | G/A | 2.92E−05 | A | 0.712 | 0.5641 |
| BICF2P1517463 | 20 | 41697094 | G/C | 2.92E−05 | C | 0.712 | 0.5641 |
| BICF2P453555 | 20 | 41709258 | T/C | 1.89E−06 | C | 0.736 | 0.5427 |
| BICF2P508868 | 20 | 41723260 | C/G | 1.75E−06 | G | 0.764 | 0.5965 |
| BICF2P372450 | 20 | 41734129 | G/A | 1.89E−06 | A | 0.736 | 0.5427 |
| BICF2P271393 | 20 | 41745091 | A/G | 1.89E−06 | G | 0.736 | 0.5427 |
| TIGRP2P274899 | 20 | 41795286 | T/C | 9.76E−07 | C | 0.764 | 0.594 |
| BICF2P716239 | 20 | 41900414 | A/G | 9.76E−07 | G | 0.764 | 0.594 |
| B1CF2P854185 | 20 | 41916205 | A/G | 2.81E−07 | G | 0.688 | 0.5128 |
| BICF2P304809 | 20 | 41924733 | T/C | 1.66E−07 | C | 0.696 | 0.5299 |
| BICF2P1310301 | 20 | 41927031 | A/G | 1.66E−07 | G | 0.696 | 0.5299 |
| BICF2P1310305 | 20 | 41930509 | A/G | 1.66E−07 | G | 0.696 | 0.5299 |
| BICF2P1231294 | 20 | 41951828 | C/T | 1.66E−07 | T | 0.696 | 0.5214 |
| BICF2P541405 | 20 | 41954052 | A/C | 1.66E−07 | C | 0.696 | 0.5299 |
| BICF2P112281 | 20 | 41991115 | G/A | 1.66E−07 | A | 0.696 | 0.5214 |
| BICF2P1185290 | 20 | 42004062 | T/C | 1.56E-08 | C | 0.704 | 0.5172 |
| BICF2S23160763 | 20 | 42071038 | C/T | 1.03E−06 | C | 0.728 | 0.5598 |
| chr20.42080147 | 20 | 42080147 | C/T | 1.09E-15 | C | 0.3733 | 0.1175 |
| BICF2P611903 | 20 | 42083608 | G/C | 3.10E−05 | G | 0.728 | 0.5598 |
| BICF2P250980 | 20 | 42095538 | A/G | 2.05E−06 | A | 0.796 | 0.6538 |
| BICF2P1241961 | 20 | 42114184 | A/G | 7.58E−07 | A | 0.764 | 0.5855 |
| BICF2P134412 | 20 | 42151061 | C/T | 6.85E−07 | C | 0.764 | 0.5872 |
| BICF2P1191632 | 20 | 42272764 | A/G | 6.47E−06 | A | 0.692 | 0.5556 |
| BICF2P927225 | 20 | 42375806 | C/T | 6.47E−06 | T | 0.692 | 0.5556 |
| TIGRP2P274941 | 20 | 42386452 | C/T | 6.47E−06 | T | 0.692 | 0.5556 |
| BICF2P476394 | 20 | 42406453 | C/T | 1.31E−05 | T | 0.8 | 0.6453 |
| BICF2P1173489 | 20 | 42415710 | A/G | 1.31E−05 | G | 0.8 | 0.641 |
| BICF2P458881 | 20 | 42477560 | C/T | 2.87E−06 | C | 0.716 | 0.5385 |
| BICF2P861824 | 20 | 42483020 | C/T | 1.02E−05 | C | 0.708 | 0.5385 |
| BICF2S22934685 | 20 | 42547825 | T/C | 5.67E−07 | T | 0.74 | 0.5299 |
| BICF2S2295117 | 20 | 42587791 | G/A | 3.09E−05 | G | 0.772 | 0.6068 |
| BICF2S23139889 | 20 | 42936673 | T/C | 3.77E−05 | C | 0.788 | 0.6453 |
| BICF2P1444805 | 20 | 42957449 | G/A | 3.48E−07 | G | 0.756 | 0.5769 |
| BICF2S2305218 | 20 | 42975776 | A/G | 2.59E−05 | G | 0.7903 | 0.6422 |
| BICF2S23324924 | 20 | 42988068 | C/T | 3.48E−07 | T | 0.756 | 0.5769 |
| BICF2S23042441 | 20 | 43709065 | G/A | 5.03E−05 | A | 0.608 | 0.4658 |
| BICF2P1256998 | 20 | 43762559 | A/C | 3.11E−05 | C | 0.612 | 0.4701 |
| BICF2P830721 | 20 | 43848341 | G/A | 5.03E−05 | A | 0.608 | 0.4658 |
| BICF2S23334554 | 20 | 43935688 | G/A | 3.80E−05 | A | 0.584 | 0.4188 |
| BICF2S23158681 | 20 | 43941778 | G/A | 3.80E−05 | A | 0.584 | 0.4188 |
| BICF2S23763114 | 20 | 44001043 | A/G | 4.02E−05 | G | 0.584 | 0.4181 |
| BICF2S22952333 | 20 | 44027026 | G/A | 3.80E−05 | A | 0.584 | 0.4188 |
| BICF2S22931382 | 20 | 44097048 | A/G | 7.28E−04 | G | 0.644 | 0.4957 |
| BICF2S23216159 | 20 | 44105651 | G/A | 3.80E−05 | A | 0.584 | 0.4188 |
| BICF2S23343399 | 20 | 44122748 | T/C | 3.80E−05 | C | 0.584 | 0.4188 |
| BICF2S23212666 | 20 | 44128697 | C/T | 3.80E−05 | T | 0.584 | 0.4188 |
| BICF2S23152344 | 20 | 44167432 | T/C | 1.40E−05 | C | 0.592 | 0.4231 |
| BICF2S22923756 | 20 | 44198701 | T/C | 1.40E−05 | C | 0.592 | 0.4231 |
| BICF2S23726023 | 20 | 44246884 | C/T | 3.80E−05 | T | 0.584 | 0.4188 |
| BICF2S23150491 | 20 | 44312048 | A/G | 3.80E−05 | G | 0.584 | 0.4188 |
| BICF2S23748153 | 20 | 44331745 | G/A | 3.80E−05 | A | 0.584 | 0.4188 |
| BICF2S23415717 | 20 | 44354720 | T/C | 5.04E−06 | C | 0.6 | 0.4231 |
| BICF2P1394766 | 20 | 44400207 | G/A | 8.66E−06 | A | 0.588 | 0.4145 |
| BICF2P861196 | 20 | 44849564 | C/T | 7.41E−04 | T | 0.62 | 0.4829 |
| BICF2S23713080 | 20 | 44941862 | A/C | 2.82E−04 | C | 0.628 | 0.5 |
| BICF2S23340206 | 20 | 44955843 | A/C | 2.82E−04 | C | 0.628 | 0.4957 |
| BICF2P1179081 | 20 | 45301965 | A/T | 4.68E−04 | T | 0.56 | 0.4231 |
| BICF2P608559 | 20 | 45311886 | G/A | 4.68E−04 | A | 0.54 | 0.4188 |
| BICF2P782456 | 20 | 45327022 | C/T | 4.68E−04 | T | 0.556 | 0.4188 |
| BICF2P911789 | 20 | 45335884 | A/G | 4.43E−04 | G | 0.556 | 0.4274 |
| BICF2P926434 | 20 | 45355933 | G/A | 4.43E−04 | A | 0.556 | 0.4274 |
| BICF2P299210 | 20 | 45359331 | T/G | 4.43E−04 | G | 0.54 | 0.4274 |
| BICF2S233350 | 20 | 45467889 | C/T | 3.58E−04 | T | 0.54 | 0.3966 |
| BICF2P696014 | 20 | 46174459 | T/A | 1.42E−04 | T | 0.42 | 0.2479 |
| BICF2P81421 | 20 | 46187197 | G/A | 1.42E−04 | G | 0.42 | 0.2436 |
| BICF2S23725316 | 20 | 46197200 | T/C | 1.45E−04 | C | 0.44 | 0.2821 |
| BICF2P716231 | 20 | 46238879 | T/G | 1.42E−04 | G | 0.432 | 0.2436 |
| B1CF2P1317092 | 20 | 46438016 | G/A | 5.09E−04 | G | 0.448 | 0.312 |
| BICF2P294403 | 20 | 46448776 | G/A | 4.97E−04 | G | 0.448 | 0.3097 |
| BICF2S23427242 | 20 | 47068232 | G/A | 2.88E−04 | A | 0.428 | 0.2821 |
| BICF2P1144529 | 20 | 47520654 | C/T | 3.04E−04 | T | 0.444 | 0.3125 |
| BICF2P787087 | 20 | 47551706 | G/A | 8.95E−05 | A | 0.444 | 0.312 |
| BICF2P1429562 | 20 | 47585373 | T/C | 8.95E−05 | C | 0.444 | 0.312 |
| BICF2P1429559 | 20 | 47588306 | A/T | 8.95E−05 | T | 0.444 | 0.312 |
| BICF2P1313482 | 20 | 47607715 | G/A | 8.95E−05 | A | 0.444 | 0.312 |
| BICF2P878447 | 20 | 47709032 | T/C | 7.88E−05 | C | 0.448 | 0.3103 |
| BICF2S23532900 | 20 | 47839318 | T/G | 3.20E−05 | T | 0.436 | 0.3077 |
| BICF2P1324128 | 20 | 47908830 | C/G | 1.17E−05 | G | 0.436 | 0.2692 |
| BICF2P951309 | 20 | 47944650 | A/C | 5.06E−06 | C | 0.436 | 0.2778 |
| BICF2P1084749 | 20 | 47963302 | G/A | 5.06E−06 | G | 0.436 | 0.2778 |
| BICF2P1050738 | 20 | 47970548 | T/C | 4.90E−06 | C | 0.436 | 0.2759 |
| BICF2P1405309 | 20 | 48077227 | T/C | 6.87E−06 | C | 0.452 | 0.3162 |
| BICF2S23510370 | 20 | 48264265 | A/G | 1.87E−04 | A | 0.492 | 0.3675 |
| BICF2P299292 | 20 | 48377580 | C/A | 2.19E−06 | A | 0.444 | 0.2692 |
| BICF2P301921 | 20 | 48599799 | C/A | 8.81E−07 | C | 0.448 | 0.2607 |
| BICF2P302160 | 20 | 48837386 | A/C | 1.74E−05 | A | 0.464 | 0.3376 |
| BICF2P800294 | 20 | 48867002 | C/T | 6.38E−04 | C | 0.504 | 0.359 |
| BICF2P1465662 | 20 | 48963283 | T/C | 5.11E−06 | T | 0.444 | 0.2607 |
| BICF2P1202229 | 20 | 49028407 | T/C | 6.35E−04 | T | 0.5 | 0.3632 |
| BICF2S23030593 | 20 | 49051702 | T/C | 8.42E−06 | T | 0.448 | 0.2906 |
| BICF2P623297 | 20 | 49201505 | A/G | 1.71E−06 | A | 0.444 | 0.2479 |
| BICF2P766049 | 20 | 49690415 | G/A | 2.17E−05 | A | 0.428 | 0.265 |
| BICF2S2376197 | 20 | 49726685 | T/C | 6.52E−05 | T | 0.448 | 0.3333 |
| BICF2G630448341 | 20 | 53017458 | T/C | 3.57E−04 | T | 0.364 | 0.2543 |

In some embodiments, the SNP may be one or more of:

i) one or more chromosome 5 SNPs,
ii) the chromosome 8 SNP TIGRP2P118921,
iii) one or more chromosome 14 SNPs, and
iv) one or more chromosome 20 SNPs, which are provided in Table 1A.

Additional chromosome 14 SNPs and chromosome 20 SNPs are provided in Table 1B. Accordingly, in some embodiments, the SNP may be one or more of the SNPs provided in Table 1B.

TABLE 1B
List of Additional SNPs associated with elevated risk of mast cell cancer

| | | | NUCLEOTIDE | | | | |
|---|---|---|---|---|---|---|---|
| | | | IDENTITY | | | Frequency | Frequency |
| | CHROMO- | | (NON- | | | risk allele | risk allele |
| SNP ID | SOME | POSITION | RISK/RISK) | SIGNIFICANCE | Ref | cases | controls |
| chr14: 14653880 | 14 | 14653880 | T/C | 8.82E−04 | T | 0.6111 | 0.4426 |
| chr14: 14666424 | 14 | 14666424 | T/C | 3.73E−05 | T | 0.7308 | 0.5244 |
| chr14: 14682089 | 14 | 14682089 | C/T | 1.22E−04 | T | 0.7812 | 0.5966 |
| chr14: 14685602 | 14 | 14685602 | A/G | 1.75E−04 | G | 0.8188 | 0.6458 |
| chr14: 14685771 | 14 | 14685771 | T/G | 7.91E−05 | G | 0.7938 | 0.6066 |
| chr20: 41512961 | 20 | 41512961 | A/C | 1.19E−04 | C | 0.5674 | 0.4148 |
| chr20: 41543010 | 20 | 41543010 | G/A | 6.33E−04 | A | 0.6403 | 0.5055 |
| chr20: 41712898 | 20 | 41712898 | G/A | 1.48E−04 | A | 0.6608 | 0.5134 |
| chr20: 41732334 | 20 | 41732334 | C/T | 2.65E−05 | T | 0.675 | 0.5108 |
| chr20: 41733976 | 20 | 41733976 | A/G | 1.65E−04 | G | 0.6655 | 0.5189 |
| chr20: 41828740 | 20 | 41828740 | C/T | 1.31E−05 | C | 0.5468 | 0.3743 |
| chr20: 41927603 | 20 | 41927603 | C/T | 1.11E−04 | T | 0.6127 | 0.4383 |
| chr20: 41933198 | 20 | 41933198 | A/G | 8.01E−05 | G | 0.6119 | 0.457 |
| chr20: 41970787 | 20 | 41970787 | A/G | 5.13E−04 | G | 0.6901 | 0.5568 |
| chr20: 41972158 | 20 | 41972158 | T/C | 3.88E−04 | C | 0.7359 | 0.6033 |
| chr20: 41972956 | 20 | 41972956 | T/C | 1.59E−05 | C | 0.6268 | 0.4574 |
| chr20: 41987996 | 20 | 41987996 | A/G | 2.36E−05 | G | 0.6232 | 0.4568 |
| chr20: 41990290 | 20 | 41990290 | T/C | 2.70E−05 | C | 0.6277 | 0.4617 |
| chr20: 41993220 | 20 | 41993220 | G/T | 3.93E−05 | T | 0.6181 | 0.4568 |
| chr20: 42060186 | 20 | 42060186 | C/T | 1.49E−06 | C | 0.5766 | 0.3846 |
| chr20: 42080147 | 20 | 42080147 | C/T | 1.23E−16 | C | 0.4028 | 0.1243 |
| chr20: 42108401 | 20 | 42108401 | G/A | 6.54E−05 | G | 0.6957 | 0.5405 |
| chr20: 42114307 | 20 | 42114307 | G/G | 4.74E−05 | G | 0.6972 | 0.5405 |
| chr20: 42115073 | 20 | 42115073 | A/G | 8.33E−05 | A | 0.6884 | 0.5351 |
| chr20: 42117345 | 20 | 42117345 | G/T | 1.37E−04 | G | 0.6879 | 0.5405 |
| chr20: 42131456 | 20 | 42131456 | G/A | 8.52E−07 | G | 0.6064 | 0.4127 |
| chr20: 42131853 | 20 | 42131853 | A/G | 6.04E−05 | A | 0.6655 | 0.5081 |
| chr20: 47886402 | 20 | 47886402 | T/C | 2.47E−05 | T | 0.3821 | 0.2297 |
| chr20: 47899650 | 20 | 47899650 | C/A | 2.12E−05 | C | 0.3811 | 0.2283 |
| chr20: 48052681 | 20 | 48052681 | T/C | 5.65E−06 | T | 0.3908 | 0.227 |
| chr20: 48056097 | 20 | 48056097 | A/G | 5.83E−06 | G | 0.1884 | 0.07065 |
| chr20: 48059078 | 20 | 48059078 | C/T | 1.41E−05 | C | 0.3854 | 0.2302 |
| chr20: 48062854 | 20 | 48062854 | A/G | 1.52E−05 | G | 0.3881 | 0.2328 |
| chr20: 48072724 | 20 | 48072724 | G/A | 6.36E−05 | G | 0.4143 | 0.265 |
| chr20: 48111692 | 20 | 48111692 | C/T | 7.23E−06 | C | 0.3873 | 0.2255 |
| chr20: 48112205 | 20 | 48112205 | C/T | 1.24E−05 | C | 0.3854 | 0.2283 |
| chr20: 48117256 | 20 | 48117256 | G/A | 6.00E−05 | G | 0.3723 | 0.2285 |
| chr20: 48158297 | 20 | 48158297 | G/C | 5.39E−04 | G | 0.4266 | 0.2962 |
| chr20: 48159029 | 20 | 48159029 | G/A | 9.57E−05 | G | 0.4414 | 0.2946 |
| chr20: 48162500 | 20 | 48162500 | A/G | 3.70E−04 | A | 0.4291 | 0.2946 |
| chr20: 48259767 | 20 | 48259767 | C/T | 7.21E−04 | C | 0.4371 | 0.3095 |
| chr20: 48260231 | 20 | 48260231 | A/G | 8.98E−04 | A | 0.4424 | 0.3155 |
| chr20: 48377580 | 20 | 48377580 | C/A | 7.91E−06 | A | 0.3944 | 0.2324 |
| chr20: 48520099 | 20 | 48520099 | C/T | 6.76E−05 | C | 0.3803 | 0.2366 |
| chr20: 48756142 | 20 | 48756142 | T/G | 1.68E−04 | T | 0.4784 | 0.3324 |
| chr20: 48756169 | 20 | 48756169 | T/C | 6.66E−04 | C | 0.4613 | 0.3306 |
| chr20: 48841374 | 20 | 48841374 | A/G | 3.11E−04 | G | 0.4321 | 0.2957 |
| chr20: 48906397 | 20 | 48906397 | C/T | 4.18E−04 | T | 0.4384 | 0.3033 |
| chr20: 49051904 | 20 | 49051904 | T/C | 6.98E−04 | T | 0.3944 | 0.2698 |
| chr20: 49687024 | 20 | 49687024 | A/G | 2.07E−05 | G | 0.3865 | 0.2324 |
| chr20: 49691940 | 20 | 49691940 | G/A | 5.04E−05 | A | 0.3671 | 0.2231 |

In some embodiments, the one or more chromosome 5 SNPs are located within chromosome coordinates Chr5:8.42-10.73 Mb. In some embodiments, the one or more chromosome 14 SNPs are located within chromosome coordinates Chr14:14.64-15.38 Mb. In some embodiments, the one or more chromosome 20 SNPs are located within chromosome coordinates Chr20:34.59-53.02 Mb.

In some embodiments, a SNP may be used in the methods described herein. In some embodiments, the method comprises:

a) analyzing genomic DNA from a canine subject for the presence of a SNP selected from:
i) one or more chromosome 5 SNPs,
ii) the chromosome 8 SNP TIGRP2P118921,
iii) one or more chromosome 14 SNPs, and
iv) one or more chromosome 20 SNPs; and

b) identifying the canine subject having one or more of the SNPs as a subject (a) at elevated risk of developing a MCC or (b) having an undiagnosed MCC.

In some embodiments, the SNP is selected from one or more chromosome 14 SNPs BICF2G630521558, BICF2G630521606, BICF2G630521619, BICF2G630521572, and

BICF2P867665. In some embodiments, the SNP is BICF2P867665. In some embodiments, the SNP is selected from one or more chromosome 20 SNPs BICF2S22934685, BICF2P1444805, BICF2P299292, BICF2P301921, and BICF2P623297. In some embodiments, the SNP is BICF2P301921. In some embodiments, the germ-line risk marker is selected from one or more chromosome 20 SNPs BICF2P304809, BICF2P1310301, BICF2P1310305, BICF2P1231294, and BICF2P1185290. In some embodiments, the germ-line risk marker is the SNP located at Ch20:4,2080,147.

It is to be understood that any number of SNPs (e.g., 1, 2, 3, 4, 5, 6, 7, 8, 9, 10, 11, 12, 13, 14, 15, 16, 17, 18, 19, or 20 or more SNPs) may be detected and/or used to identify a subject.

### Risk Haplotypes

In some embodiments, a germ-line risk marker is a risk haplotype. A risk haplotype, as used herein, is a chromosomal region containing at least one mutation that correlates with the presence of or likelihood of developing MCC in a subject. A risk haplotype is detected or identified by one or more mutations. For example, a risk haplotype may be a chromosomal region with boundaries that are defined by two or more SNPs that are in linkage disequilibrium and correlate with the presence of or likelihood of developing MCC in a subject. Such SNPs may themselves be disease-causative or may, alternatively or additionally, be indicators of other mutations (either germ-line mutations or somatic mutations) present in the chromosomal region of the risk haplotype that correlate with or cause MCC in a subject. Thus, other mutations within the risk haplotype may correlate with presence of or likelihood of developing MCC in a subject and are contemplated for use in the methods herein. Accordingly, in some embodiments, methods described herein comprise use and/or detection of a risk haplotype. In some embodiments, the risk haplotype is selected from:

a risk haplotype having chromosome coordinates Chr5:8.42-10.73 Mb,

a risk haplotype having chromosome coordinates Chr14:14.64-14.76 Mb,

a risk haplotype having chromosome coordinates Chr20:41.51-42.12 Mb,

a risk haplotype having chromosome coordinates Chr20:41.70-42.59 Mb, or

a risk haplotype having chromosome coordinates Chr20:47.06-49.70 Mb.

Any chromosomal coordinates described herein are meant to be inclusive (i.e., include the boundaries of the chromosomal coordinates). In some embodiments, the risk haplotype may include additional chromosomal regions flanking those chromosomal regions described above, e.g., an additional 0.1, 0.5, 1, 2, 3, 4 or 5 Mb. In some embodiments, the risk haplotype may be a shortened chromosomal region than those chromosomal regions described above, e.g., 0.1, 0.5, or 1 Mb fewer than the chromosomal regions described above.

Any mutation of any size located within or spanning the chromosomal boundaries of a risk haplotype is contemplated herein for detection of a risk haplotype, e.g., a SNP, a deletion, an inversion, a translocation, or a duplication. In some embodiments, the risk haplotype is detected by analyzing the chromosomal region of the risk haplotype for the presence of a SNP. In some embodiments, a SNP in risk haplotype is a SNP described in Table 2. Table 2 provides exemplary SNPs within risk haplotypes on chromosomes 5, 14 and 20. Table 2 provides the non-risk and risk nucleotide for each SNP. The “REF” column of Table 2 refers to the nucleotide identity present in the Boxer reference genome. The risk nucleotide is the nucleotide that is associated with elevated risk of developing a MCC or having an undiagnosed MCC. It is to be understood that other SNPs not listed in Table 2 but located within the risk haplotype coordinates on chromosome 5, 14 and 20 above are also contemplated herein.

TABLE 2
SNPs located in risk haplotypes associated
with elevated risk of mast cell cancer

| | | | NUCLEOTIDE | |
|---|---|---|---|---|
| | | | IDENTITY | |
| | CHROMO- | | (NON- | |
| SNP ID | SOME | POSITION | RISK/RISK) | REF |
| BICF2P807873 | 5 | 8428475 | A/G | G |
| BICF2P778319 | 5 | 8431406 | T/C | C |
| BICF2P547394 | 5 | 8487193 | A/G | G |
| BICF2P1347656 | 5 | 9397630 | A/T | T |
| BICF2S2331073 | 5 | 10667930 | T/C | T |
| BICF2S23025903 | 5 | 10709446 | A/G | G |
| BICF2S23519930 | 5 | 10728844 | G/A | A |
| BICF2G630521558 | 14 | 14644897 | T/C | C |
| BICF2G630521572 | 14 | 14670361 | C/T | T |
| BICF2G630521606 | 14 | 14682089 | C/T | T |
| BICF2G630521619 | 14 | 14685543 | T/C | C |
| BICF2P867665 | 14 | 14714009 | T/G | T |
| TIGRP2P186605 | 14 | 14727905 | A/G | G |
| BICF2G630521678 | 14 | 14740313 | G/A | G |
| BICF2G630521681 | 14 | 14743663 | T/C | T |
| BICF2G630521696 | 14 | 14756089 | A/G | A |
| BICF2P453555 | 20 | 41709258 | T/C | C |
| BICF2P372450 | 20 | 41734129 | G/A | A |
| BICF2P271393 | 20 | 41745091 | A/G | G |
| BICF2S22934685 | 20 | 42547825 | T/C | T |
| BICF2S2295117 | 20 | 42587791 | G/A | G |
| BICF2S23427242 | 20 | 47068232 | G/A | A |
| BICF2P1144529 | 20 | 47520654 | C/T | T |
| BICF2P787087 | 20 | 47551706 | G/A | A |
| BICF2P1429562 | 20 | 47585373 | T/C | C |
| BICF2P1429559 | 20 | 47588306 | A/T | T |
| BICF2P1313482 | 20 | 47607715 | G/A | A |
| BICF2P878447 | 20 | 47709032 | T/C | C |
| BICF2S23532900 | 20 | 47839318 | T/G | T |
| BICF2P1324128 | 20 | 47908830 | C/G | G |
| BICF2P951309 | 20 | 47944650 | A/C | C |
| BICF2P1084749 | 20 | 47963302 | G/A | G |
| BICF2P1050738 | 20 | 47970548 | T/C | C |
| BICF2P1405309 | 20 | 48077227 | T/C | C |
| BICF2P299292 | 20 | 48377580 | C/A | A |
| BICF2P301921 | 20 | 48599799 | C/A | C |
| BICF2P1465662 | 20 | 48963283 | T/C | T |
| BICF2S23030593 | 20 | 49051702 | T/C | T |
| BICF2P623297 | 20 | 49201505 | A/G | A |
| BICF2P766049 | 20 | 49690415 | G/A | A |
| BICF2P807873 | 5 | 8428475 | A/G | G |
| BICF2P778319 | 5 | 8431406 | T/C | C |
| BICF2P547394 | 5 | 8487193 | A/G | G |
| BICF2P1347656 | 5 | 9397630 | A/T | T |
| BICF2S2331073 | 5 | 10667930 | T/C | T |
| BICF2S23025903 | 5 | 10709446 | A/G | G |
| BICF2S23519930 | 5 | 10728844 | G/A | A |
| BICF2G630521558 | 14 | 14644897 | T/C | C |
| BICF2G630521572 | 14 | 14670361 | C/T | T |
| BICF2G630521606 | 14 | 14682089 | C/T | T |
| BICF2G630521619 | 14 | 14685543 | T/C | C |

In some embodiments a risk haplotype can be used in the methods described herein. In some embodiments, the method comprises:

analyzing genomic DNA from a canine subject for the presence of a risk haplotype selected from:
a risk haplotype having chromosome coordinates Chr5:8.42-10.73 Mb,
a risk haplotype having chromosome coordinates Chr14:14.64-14.76 Mb,
a risk haplotype having chromosome coordinates Chr20:41.51-42.12 Mb,
a risk haplotype having chromosome coordinates Chr20:41.70-42.59 Mb, and
a risk haplotype having chromosome coordinates Chr20:47.06-49.70 Mb; and

identifying a canine subject having the risk haplotype as a subject (a) at elevated risk of developing a MCC or (b) having an undiagnosed MCC. In some embodiments, the risk haplotype is selected from
the risk haplotype having chromosome coordinates Chr14:14.64-14.76 Mb,
the risk haplotype having chromosome coordinates Chr20:41.51-42.12 Mb,
the risk haplotype having chromosome coordinates Chr20:41.70-42.59 Mb, and
the risk haplotype having chromosome coordinates Chr20:47.06-49.70 Mb.

In some embodiments, the risk haplotype is the risk haplotype having chromosome coordinates Chr14:14.64-14.76 Mb. In some embodiments, the risk haplotype is the risk haplotype having chromosome coordinates Chr20:41.51-42.12 Mb. In some embodiments, the risk haplotype is the risk haplotype having chromosome coordinates Chr20:41.70-42.59 Mb or the risk haplotype having chromosome coordinates Chr20:47.06-49.70 Mb. In some embodiments, the risk haplotype is the risk haplotype having chromosome coordinates Chr20:47.06-49.70 Mb

It is to be understood that any number of mutations (e.g., 1, 2, 3, 4, 5, 6, 7, 8, 9, 10, 11, 12, 13, 14, 15, 16, 17, 18, 19, or 20 or more mutations) can exist within each risk haplotype. It is also to be understood that not all mutations within the risk haplotype must be detected in order to determine that the risk haplotype is present. For example, one mutation may be used to detect the presence of a risk haplotype. In another example, two or more mutations may be used to detect the presence of a risk haplotype. It is also to be understood that subject identification may involve any number of risk haplotypes (e.g., 1, 2, 3, 4, or 5 risk haplotypes).

In some embodiments, the presence of a risk haplotype is determined by detecting one or more SNPs within the chromosomal coordinates of the risk haplotype. In some embodiments, the presence of the risk haplotype is detected by analyzing the genomic DNA for the presence of a SNP is selected from:

(a) Chr5:8.42-10.73 Mb SNPs BICF2P807873, BICF2P778319, BICF2P547394, BICF2P1347656, BICF2S2331073, BICF2S23025903, and BICF2S23519930,

(b) Chr14:14.64-14.76 Mb SNPs BICF2G630521558, BICF2G630521572, BICF2G630521606, BICF2G630521619, BICF2P867665, TIGRP2P186605, BICF2G630521678, BICF2G630521681, and BICF2G630521696,

(c) Chr20:41.51-42.12 Mb SNPs BICF2P453555, BICF2P372450, BICF2P271393, BICF2S22934685, BICF2S2295117,

(d) Chr20:41.70-42.59 Mb SNPs BICF2P453555, BICF2P372450, BICF2P271393, BICF2S22934685, BICF2S2295117, and

(e) Chr20:47.06-49.70 Mb SNPs BICF2P327134, BICF2P854185, BICF2P304809, BICF2P1310301, BICF2P1310305, BICF2P1231294, BICF2P541405, BICF2P112281, BICF2P1185290, and BICF2P1241961.

It is to be understood that any number of SNPs (e.g., 1, 2, 3, 4, 5, 6, 7, 8, 9, 10, 11, 12, 13, 14, 15, 16, 17, 18, 19, or 20 or more SNPs) in any number of risk haplotypes (e.g., 1, 2, 3, 4, or 5 risk haplotypes) may be used. In some embodiments, a subset or all SNPs located in a risk haplotype in Table 2 are used (e.g., a subset or all 9 SNPs in the risk haplotype having chromosome coordinates Chr14:14.64-14.76 Mb, and/or a subset or all 15 SNPS in the risk haplotype having chromosome coordinates Chr20:41.70-42.59 Mb, and/or a subset or all 20 SNPs in the risk haplotype having chromosome coordinates Chr20:47.06-49.70 Mb).

### Genes

In some embodiments, a germ-line risk marker is a mutation in a gene. As used herein, a gene includes both coding and non-coding sequences. As such, a gene includes any regulatory sequences (e.g., any promoters, enhancers, or suppressors, either adjacent to or far from the coding sequence) and any coding sequences. In some embodiments, the gene is contained within, near, or spanning the boundaries of a risk haplotype as described herein. In some embodiments, a mutation, such as a SNP, is contained within or near the gene. In some embodiments, the gene is within 1000 Kb, 900 Kb, 800 Kb, 700 Kb, 600 Kb, 500 Kb, 400 Kb, 300 Kb, 200 Kb, or 100 Kb of a SNP as described herein. In some embodiments, the gene is within 500 Kb of a SNP as described herein, such as TIGRP2P118921. In some embodiments, the mutation is present in a gene selected from:

one or more genes located within a risk haplotype having chromosome coordinates Chr5:8.42-10.73 Mb,

one or more genes within 500 Kb of TIGRP2P118921 on chromosome 8,

one or more genes located within a risk haplotype having chromosome coordinates Chr14:14.64-14.76 Mb,

one or more genes located within a risk haplotype having chromosome coordinates Chr20:41.51-42.12 Mb,

one or more genes located within a risk haplotype having chromosome coordinates Chr20:41.70-42.59 Mb, and

one or more genes located within a risk haplotype having chromosome coordinates Chr20:47.06-49.70 Mb.

The mapped genes located within the risk haplotypes on chromosome 5, 8, 14 and 20 are described in Table 3. The Ensembl gene identifiers are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The Ensembl gene ID provided for each gene can be used to determine the sequence of the gene, as well as associated transcripts and proteins, by inputting the Ensemble ID into the Ensemble database (Ensembl release 70).

TABLE 3
Genes present in chromosomal regions associated
with elevated risk of mast cell cancer

| | Ensembl gene ID, | Ensemble gene ID, |
|---|---|---|
| Gene | Canine | Human |
| SLC25A42 | ENSCAFG00000014386 | ENSG00000181035 |
| ARMC6 | ENSCAFG00000014404 | ENSG00000105676 |
| SUGP2 | ENSCAFG00000014431 | ENSG00000064607 |
| HOMER3 | ENSCAFG00000014475 | ENSG00000051128 |
| DDX49 | ENSCAFG00000014512 | ENSG00000105671 |
| CERS1 | ENSCAFG00000023156 | ENSG00000223802 |
| No gene name | ENSCAFG00000014540 | N/A |
| UPF1 | ENSCAFG00000014578 | ENSG00000005007 |
| COMP | ENSCAFG00000014616 | ENSG00000105664 |
| No gene name | ENSCAFG00000014647 | N/A |
| 5S_rRNA | ENSCAFG00000022146 | N/A |
| U6 | ENSCAFG00000027972 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| KLHL26 | ENSCAFG00000014671 | ENSG00000167487 |
| TMEM59L | ENSCAFG00000014687 | ENSG00000105696 |
| CRLF1 | ENSCAFG00000014698 | ENSG00000006016 |
| C19orf60 | ENSCAFG00000014713 | ENSG00000006015 |
| RL40_CANFA | ENSCAFG00000014723 | N/A |
| KXD1 | ENSCAFG00000014727 | ENSG00000105700 |
| FKBP8 | ENSCAFG00000014742 | ENSG00000105701 |
| ELL | ENSCAFG00000014770 | ENSG00000105656 |
| ISYNA1 | ENSCAFG00000014817 | ENSG00000105655 |
| SSBP4 | ENSCAFG00000014862 | ENSG00000130511 |
| LRRC25 | ENSCAFG00000014879 | ENSG00000175489 |
| GDF15 | ENSCAFG00000014882 | ENSG00000130513 |
| No gene name | ENSCAFG00000014886 | N/A |
| PGPEP1 | ENSCAFG00000014891 | ENSG00000130517 |
| LSM4 | ENSCAFG00000014900 | ENSG00000130520 |
| JUND | ENSCAFG00000023338 | ENSG00000130522 |
| No gene name | ENSCAFG00000029989 | N/A |
| KIAA1683 | ENSCAFG00000014907 | ENSG00000130518 |
| PDE4C | ENSCAFG00000014928 | ENSG00000105650 |
| RAB3A | ENSCAFG00000014945 | ENSG00000105649 |
| MPV17L2 | ENSCAFG00000014954 | ENSG00000254858 |
| IFI30 | ENSCAFG00000014956 | ENSG00000216490 |
| PIK3R2 | ENSCAFG00000014978 | ENSG00000105647 |
| MAST3 | ENSCAFG00000015009 | ENSG00000099308 |
| IL12RB1 | ENSCAFG00000015028 | ENSG00000096996 |
| ARRDC2 | ENSCAFG00000015088 | ENSG00000105643 |
| KCNN1 | ENSCAFG00000015092 | ENSG00000105642 |
| No gene name | ENSCAFG00000015098 | N/A |
| No gene name | ENSCAFG00000024472 | N/A |
| SLC5A5 | ENSCAFG00000015051 | ENSG00000105641 |
| No gene name | ENSCAFG00000015122 | N/A |
| SNORA68 | ENSCAFG00000026322 | ENSG00000251715 |
| | | ENSG00000252458 |
| | | ENSG00000201407 |
| | | ENSG00000212565 |
| | | ENSG00000201388 |
| | | ENSG00000207166 |
| JAK3 | ENSCAFG00000015159 | ENSG00000105639 |
| INSL3 | ENSCAFG00000032526 | ENSG00000248099 |
| B3GNT3 | ENSCAFG00000015192 | ENSG00000179913 |
| FCHO1 | ENSCAFG00000015212 | ENSG00000130475 |
| MAP1S | ENSCAFG00000015229 | ENSG00000130479 |
| No gene name | ENSCAFG00000024064 | N/A |
| No gene name | ENSCAFG00000028977 | N/A |
| U6 | ENSCAFG00000026172 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| GLT25D1 | ENSCAFG00000031738 | ENSG00000130309 |
| FAM129C | ENSCAFG00000015256 | ENSG00000167483 |
| PGLS | ENSCAFG00000015270 | ENSG00000130313 |
| SLC27A1 | ENSCAFG00000015315 | ENSG00000130304 |
| NXNL1 | ENSCAFG00000015327 | ENSG00000171773 |
| TMEM221 | ENSCAFG00000015329 | ENSG00000188051 |
| FAM125A | ENSCAFG00000015332 | ENSG00000141971 |
| BST2 | ENSCAFG00000031353 | ENSG00000130303 |
| PLVAP | ENSCAFG00000015337 | ENSG00000130300 |
| GTPBP3 | ENSCAFG00000015378 | ENSG00000130299 |
| ANO8 | ENSCAFG00000015416 | ENSG00000074855 |
| DDA1 | ENSCAFG00000031251 | ENSG00000130311 |
| MRPL34 | ENSCAFG00000028802 | ENSG00000130312 |
| ABHD8 | ENSCAFG00000015430 | ENSG00000127220 |
| ANKLE1 | ENSCAFG00000015434 | ENSG00000160117 |
| BABAM1 | ENSCAFG00000015454 | ENSG00000105393 |
| USHBP1 | ENSCAFG00000015462 | ENSG00000130307 |
| NR2F6 | ENSCAFG00000015487 | ENSG00000160113 |
| OCEL1 | ENSCAFG00000015500 | ENSG00000099330 |
| USE1 | ENSCAFG00000015513 | ENSG00000053501 |
| MYO9B | ENSCAFG00000015532 | ENSG00000099331 |
| HAUS8 | ENSCAFG00000015551 | ENSG00000131351 |
| PPDPF | ENSCAFG00000015555 | ENSG00000125534 |
| CPAMD8 | ENSCAFG00000015590 | ENSG00000160111 |
| F2RL3 | ENSCAFG00000015606 | ENSG00000127533 |
| SIN3B | ENSCAFG00000015616 | ENSG00000127511 |
| NWD1 | ENSCAFG00000015626 | ENSG00000188039 |
| TMEM38A | ENSCAFG00000030694 | ENSG00000072954 |
| C19orf42 | ENSCAFG00000015643 | ENSG00000214046 |
| MED26 | ENSCAFG00000015648 | ENSG00000105085 |
| SLC35E1 | ENSCAFG00000015651 | ENSG00000127526 |
| CHERP | ENSCAFG00000015671 | ENSG00000085872 |
| C19orf44 | ENSCAFG00000015691 | ENSG00000105072 |
| CALR3 | ENSCAFG00000015694 | ENSG00000141979 |
| EPS15L1 | ENSCAFG00000015735 | ENSG00000127527 |
| AP1M1 | ENSCAFG00000015762 | ENSG00000072958 |
| CIB3 | ENSCAFG00000015775 | ENSG00000141977 |
| HSH2D | ENSCAFG00000015778 | ENSG00000196684 |
| RAB8A_CANFA | ENSCAFG00000015782 | ENSG00000167461 |
| TPM4 | ENSCAFG00000015796 | ENSG00000167460 |
| No gene name | ENSCAFG00000028520 | N/A |
| No gene name | ENSCAFG00000031088 | N/A |
| No gene name | ENSCAFG00000015814 | N/A |
| No gene name | ENSCAFG00000028482 | N/A |
| No gene name | ENSCAFG00000030903 | N/A |
| No gene name | ENSCAFG00000028658 | N/A |
| No gene name | ENSCAFG00000015833 | N/A |
| No gene name | ENSCAFG00000030089 | N/A |
| No gene name | ENSCAFG00000023401 | N/A |
| No gene name | ENSCAFG00000015931 | N/A |
| CYP4F22 | ENSCAFG00000023053 | ENSG00000171954 |
| HYAL4 | ENSCAFG00000001768 | ENSG00000106302 |
| HYALP1 | ENSCAFG00000024436 | ENSG00000228211 |
| SPAM1/PH20 | ENSCAFG00000001765 | ENSG00000106304 |
| CYB561D2 | ENSCAFG00000010581 | ENSG00000114395 |
| No gene name | ENSCAFG00000010754 | N/A |
| No gene name | ENSCAFG00000010719 | N/A |
| GNAI2 | ENSCAFG00000010740 | ENSG00000114353 |
| | | ENSG00000263156 |
| TUSC2 | ENSCAFG00000010651 | ENSG00000262485 |
| | | ENSG00000114383 |
| RASSF1 | ENSCAFG00000010627 | ENSG00000263005 |
| | | ENSG00000068028 |
| ZMYND10 | ENSCAFG00000010609 | ENSG00000004838 |
| NPRL2 | ENSCAFG00000010590 | ENSG00000114388 |
| CYB561D2 | ENSCAFG00000010581 | ENSG00000114395 |
| TMEM115 | ENSCAFG00000010578 | ENSG00000126062 |
| C3orf18 | ENSCAFG00000010303 | ENSG00000088543 |
| HEMK1 | ENSCAFG00000010296 | ENSG00000114735 |
| CISH | ENSCAFG00000010293 | ENSG00000114737 |
| MAPKAPK3 | ENSCAFG00000010281 | ENSG00000114738 |
| RPS6KA5 | ENSCAFG00000017543 | ENSG00000100784 |
| GPR68 | ENSCAFG00000017555 | ENSG00000119714 |
| CCDC88C | ENSCAFG00000017561 | ENSG00000015133 |
| SMEK1 | ENSCAFG00000017570 | ENSG00000100796 |
| 5S_rRNA | ENSCAFG00000021972 | N/A |
| U6 | ENSCAFG00000030334 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| TMEM251 | ENSCAFG00000017588 | ENSG00000153485 |
| C14orf142 | ENSCAFG00000032108 | ENSG00000170270 |
| | ENSCAFG00000017591 | N/A |
| BTBD7 | ENSCAFG00000017600 | ENSG00000011114 |
| U6 | ENSCAFG00000021074 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| 7SK | ENSCAFG00000028390 | N/A |
| UNC79 | ENSCAFG00000017606 | ENSG00000133958 |
| U6 | ENSCAFG00000027623 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| PRIMA1 | ENSCAFG00000032722 | ENSG00000175785 |
| FAM181A | ENSCAFG00000017609 | ENSG00000140067 |
| ASB2 | ENSCAFG00000017612 | ENSG00000100628 |
| No gene name | ENSCAFG00000017617 | N/A |
| OTUB2 | ENSCAFG00000017619 | ENSG00000089723 |
| DDX24 | ENSCAFG00000017624 | ENSG00000089737 |
| IFI27 | ENSCAFG00000017632 | ENSG00000165949 |
| PPP4R4 | ENSCAFG00000017636 | ENSG00000119698 |
| SERPINA6 | ENSCAFG00000024698 | ENSG00000170099 |
| SERPINA1 | ENSCAFG00000017646 | ENSG00000197249 |
| SERPINA11 | ENSCAFG00000024668 | ENSG00000186910 |
| C9E9X8_CANFA | ENSCAFG00000017659 | N/A |
| SERPINA9 | ENSCAFG00000024137 | ENSG00000170054 |
| SERPINA12 | ENSCAFG00000017661 | ENSG00000165953 |
| SERPINA4 | ENSCAFG00000023610 | ENSG00000100665 |
| SERPINA5 | ENSCAFG00000029000 | ENSG00000188488 |
| SERPINA3 | ENSCAFG00000017675 | ENSG00000196136 |
| GSC | ENSCAFG00000017684 | ENSG00000133937 |
| U6 | ENSCAFG00000032705 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| ARHGAP32 | ENSCAFG00000010235 | ENSG00000134909 |
| KCNJ5 | ENSCAFG00000010255 | ENSG00000120457 |
| KCNJ1 | ENSCAFG00000010259 | ENSG00000151704 |
| FLI1 | ENSCAFG00000032412 | ENSG00000151702 |
| A1XFH2_CANFA | ENSCAFG00000010304 | N/A |
| U6 | ENSCAFG00000032431 | ENSG00000201654 |
| | | ENSG00000202337 |
| | | ENSG00000206932 |
| | | ENSG00000206965 |
| | | ENSG00000207041 |
| | | ENSG00000207357 |
| | | ENSG00000207507 |
| MAPKAPK3 | ENSCAFG00000010281 | ENSG00000114738 |
| CISH | ENSCAFG00000010293 | ENSG00000114737 |
| HEMK1 | ENSCAFG00000010296 | ENSG00000114735 |
| C3orf18 | ENSCAFG00000010303 | ENSG00000088543 |
| CACNA2D2 | ENSCAFG00000010431 | ENSG00000007402 |
| TMEM115 | ENSCAFG00000010578 | ENSG00000126062 |
| CYB561D2 | ENSCAFG00000010581 | ENSG00000114395 |
| NPRL2 | ENSCAFG00000010590 | ENSG00000114388 |
| ZMYND10 | ENSCAFG00000010609 | ENSG00000004838 |
| RASSF1 | ENSCAFG00000010627 | ENSG00000263005 |
| | | ENSG00000068028 |
| TUSC2 | ENSCAFG00000010651 | ENSG00000262485 |
| | | ENSG00000114383 |
| HYAL2 | ENSCAFG00000010657 | ENSG00000261921 |
| | | ENSG00000068001 |
| HYAL1 | ENSCAFG00000010599 | ENSG00000114378 |
| | | ENSG00000262208 |
| HYAL3 | ENSCAFG00000010672 | ENSG00000186792 |
| | | ENSG00000261855 |
| C3orf45 | ENSCAFG00000010695 | ENSG00000179564 |
| | | ENSG00000261869 |
| No gene name | ENSCAFG00000010719 | N/A |
| GNAI2_CANFA | ENSCAFG00000010740 | ENSG00000114353 |
| | | ENSG00000263156 |
| No gene name | ENSCAFG00000010754 | N/A |
| GNAT1_CANFA | ENSCAFG00000010764 | ENSG00000114349 |
| SEMA3F | ENSCAFG00000010804 | ENSG00000001617 |
| RBM5 | ENSCAFG00000010866 | ENSG00000003756 |
| RBM6 | ENSCAFG00000010914 | ENSG00000004534 |
| MON1A | ENSCAFG00000010939 | ENSG00000164077 |
| No gene name | ENSCAFG00000010974 | N/A |
| CAMKV | ENSCAFG00000011008 | ENSG00000164076 |
| TRAIP | ENSCAFG00000011057 | ENSG00000183763 |
| UBA7 | ENSCAFG00000011164 | ENSG00000182179 |
| FAM212A | ENSCAFG00000031572 | ENSG00000185614 |
| CDHR4 | ENSCAFG00000029789 | ENSG00000187492 |
| IP6K1 | ENSCAFG00000011226 | ENSG00000176095 |
| GMPPB | ENSCAFG00000023755 | ENSG00000173540 |
| RNF123 | ENSCAFG00000011290 | ENSG00000164068 |
| AMIGO3 | ENSCAFG00000011248 | ENSG00000176020 |
| No gene name | ENSCAFG00000011411 | N/A |
| APEH | ENSCAFG00000011449 | ENSG00000164062 |
| DOCK3 | ENSCAFG00000010229 | ENSG00000088538 |
| | | ENSG00000260587 |
| No gene name | ENSCAFG00000010275 | N/A |
| MAPKAPK3 | ENSCAFG00000010281 | ENSG00000114738 |
| CISH | ENSCAFG00000010293 | ENSG00000114737 |
| HEMK1 | ENSCAFG00000010296 | ENSG00000114735 |
| C3orf18 | ENSCAFG00000010303 | ENSG00000088543 |
| CACNA2D2 | ENSCAFG00000010431 | ENSG00000007402 |
| TMEM115 | ENSCAFG00000010578 | ENSG00000126062 |
| CYB561D2 | ENSCAFG00000010581 | ENSG00000114395 |
| NPRL2 | ENSCAFG00000010590 | ENSG00000114388 |
| ZMYND10 | ENSCAFG00000010609 | ENSG00000004838 |
| RASSF1 | ENSCAFG00000010627 | ENSG00000263005 |
| | | ENSG00000068028 |
| TUSC2 | ENSCAFG00000010651 | ENSG00000262485 |
| | | ENSG00000114383 |
| HYAL2 | ENSCAFG00000010657 | ENSG00000261921 |
| | | ENSG00000068001 |
| HYAL1 | ENSCAFG00000010599 | ENSG00000114378 |
| | | ENSG00000262208 |
| HYAL3 | ENSCAFG00000010672 | ENSG00000186792 |
| | | ENSG00000261855 |
| C3orf45 | ENSCAFG00000010695 | ENSG00000179564 |
| | | ENSG00000261869 |
| No gene name | ENSCAFG00000010719 | N/A |
| GNAI2_CANFA | ENSCAFG00000010740 | ENSG00000114353 |
| | | ENSG00000263156 |
| No gene name | ENSCAFG00000010754 | N/A |
| TMEM229A | ENSCAFG00000001762 | ENSG00000234224 |

No gene name = no known gene name available;
N/A = no identified or known corresponding human gene.

In some embodiments, a mutation in a gene is used in the methods described herein. In some embodiments, the method comprises:

analyzing genomic DNA from a canine subject for the presence of a mutation in a gene selected from
one or more genes located within a risk haplotype having chromosome coordinates Chr5:8.42-10.73 Mb,
one or more genes within 500 Kb of TIGRP2P118921 on chromosome 8,
one or more genes located within a risk haplotype having chromosome coordinates Chr14:14.64-14.76 Mb,
one or more genes located within a risk haplotype having chromosome coordinates Chr20:41.51-42.12 Mb,
one or more genes located within a risk haplotype having chromosome coordinates Chr20:41.70-42.59 Mb, and
one or more genes located within a risk haplotype having chromosome coordinates Chr20:47.06-49.70 Mb, and

identifying a canine subject having the mutation as a subject (a) at elevated risk of developing a MCC or (b) having an undiagnosed MCC.

Any number of mutations (e.g., 1, 2, 3, 4, 5, 6, 7, 8, 9, 10, 11, 12, 13, 14, 15, 16, 17, 18, 19, or 20 or more mutations) in any number of genes (e.g., 1, 2, 3, 4, 5, 6, 7, 8, 9, 10, 11, 12, 13, 14, 15, 16, 17, 18, 19, or 20 or more genes) are contemplated.

In some embodiments, the gene is selected from one or more genes located within a risk haplotype having chromosome coordinates Chr14:14.64-14.76 Mb. In some embodiments, the gene is selected from SPAM1, HYAL4, and HYALP1. In some embodiments, the gene is selected from one or more genes located within a risk haplotype having chromosome coordinates Chr20:41.51-42.12 Mb or one or more genes located within a risk haplotype having chromosome coordinates Chr20:47.06-49.70 Mb. In some embodiments, the gene is selected from one or more genes located within a risk haplotype having chromosome coordinates Chr20:47.06-49.70 Mb. In some embodiments, the gene is selected from one or more genes located within a risk haplotype having chromosome coordinates Chr20:41.51-42.12 Mb. In some embodiments, the gene is selected from DOCK3, ENSCAFG00000010275, MAPKAPK3, CISH, HEMK1, C3orf18, CACNA2D2, TMEM115, NPRL2, ZMYND10, RASSF1, TUSC2, HYAL2, HYAL1, HYAL3, C3orf45, ENSCAFG00000010719, GNAI2_CANFA, and ENSCAFG00000010754. In some embodiments, the gene is selected from MAPKAPK3, CISH, HEMK1, C3orf18, CACNA2D2, TMEM115, CYB561D2, NPRL2, ZMYND10, RASSF1, TUSC2, HYAL2, HYAL1, HYAL3, C3oef45, GNAI2, ENSCAFG00000010719, and ENSCAFG00000010754. In some embodiments, the gene is GNAI2. In some embodiments, the gene is selected from HYAL1, HYAL2, HYAL3, SPAM1, HYAL4, HYALP1, and TMEM229A. In some embodiments, the gene is TMEM229A.

Aspects of the invention are based in part on the discovery of a correlation of risk haplotypes containing hyaluronidase genes with MCC. In some embodiments, a mutation in a hyaluronidase gene is used in the methods described herein. In some embodiments, the method comprises:

analyzing genomic DNA from a subject for the presence of a mutation in a hyaluronidase gene; and

identifying a subject having the mutation as a subject (a) at elevated risk of developing a MCC or (b) having an undiagnosed MCC. In some embodiments, the subject is a canine subject. In some embodiments, the subject is a human subject. In some embodiments, the hyaluronidase gene is selected from HYAL1, HYAL2, HYAL3, SPAM1, HYAL4, and HYALP1.

In some embodiments, hyaluronidase activity may be used in the methods described herein. Hyaluronidase activity may be determined, e.g., by measuring a level of HA or hyaluronidase activity. In some embodiments, the method comprises:

analyzing hyaluronidase activity in a biological sample from a subject; and

identifying a subject having decreased hyaluronidase activity as a subject (a) at elevated risk of developing a MCC or (b) having an undiagnosed MCC.

Hyaluronidase activity may be analyzed directly, e.g., using enzymatic assays, or indirectly, e.g., by measuring levels of HA. Exemplary hyaluronidase enzymatic assays are commercially available from Amsbio. Levels of HA may be determined using ELISA based methods to detect HA content in a biological sample. Commercial hyaluronic acid ELISA kits are available from Echelon and Corgenix.

The genes described herein can also be used to identify a subject at risk of or having undiagnosed MCC, where the subject is any of a variety of animal subjects including but not limited to human subjects. In some embodiments, the method, comprises analyzing genomic DNA in a sample from a subject for presence of a mutation in a gene selected from

one or more genes located within a risk haplotype having chromosome coordinates Chr5:8.42-10.73 Mb, or an orthologue of such a gene,

one or more genes within 500 Kb of TIGRP2P118921 on chromosome 8,

one or more genes located within a risk haplotype having chromosome coordinates Chr14:14.64-14.76 Mb, or an orthologue of such a gene,

one or more genes located within a risk haplotype having chromosome coordinates Chr20:41.51-42.12 Mb, or an orthologue of such a gene,

one or more genes located within a risk haplotype having chromosome coordinates Chr20:41.70-42.59 Mb, or an orthologue of such a gene, and

one or more genes located within a risk haplotype having chromosome coordinates Chr20:47.06-49.70 Mb, or an orthologue of such a gene; and

identifying a subject having the mutation as a subject (a) at elevated risk of developing MCC or (b) having an undiagnosed MCC. In some embodiments, the subject is a human subject. In some embodiments, the subject is a canine subject. An orthologue of a gene may be, e.g., a human gene as identified in Table3. In some embodiments, an orthologue of a gene has a sequence that is 70%, 75%, 80%, 85%, 90%, 95%, or 99% or more homologous to a sequence of the gene.

### Genome Analysis Methods

Some methods provided herein comprise analyzing genomic DNA. In some embodiments, analyzing genomic DNA comprises carrying out a nucleic acid-based assay, such as a sequencing-based assay or a hybridization based assay. In some embodiments, the genomic DNA is analyzed using a single nucleotide polymorphism (SNP) array. In some embodiments, the genomic DNA is analyzed using a bead array. Methods of genetic analysis are known in the art. Examples of genetic analysis methods and commercially available tools are described below.

Affymetrix:

The Affymetrix SNP 6.0 array contains over 1.8 million SNP and copy number probes on a single array. The method utilizes at a simple restriction enzyme digestion of 250 ng of genomic DNA, followed by linker-ligation of a common adaptor sequence to every fragment, a tactic that allows multiple loci to be amplified using a single primer complementary to this adaptor. Standard PCR then amplifies a predictable size range of fragments, which converts the genomic DNA into a sample of reduced complexity as well as increases the concentration of the fragments that reside within this predicted size range. The target is fragmented, labeled with biotin, hybridized to microarrays, stained with streptavidin-phycoerythrin and scanned. To support this method, Affymetrix Fluidics Stations and integrated GS-3000 Scanners can be used.

Illumina Infinium:

Examples of commercially available Infinium array options include the 660W-Quad (>660,000 probes), the 1MDuo (over 1 million probes), and the custom iSelect (up to 200,000 SNPs selected by user). Samples begin the process with a whole genome amplification step, then 200 ng is transferred to a plate to be denatured and neutralized, and finally plates are incubated overnight to amplify. After amplification the samples are enzymatically fragmented using end-point fragmentation. Precipitation and resuspension clean up the DNA before hybridization onto the chips. The fragmented, resuspended DNA samples are then dispensed onto the appropriate BeadChips and placed in the hybridization oven to incubate overnight. After hybridization the chips are washed and labeled nucleotides are added to extend the primers by one base. The chips are immediately stained and coated for protection before scanning. Scanning is done with one of the two Illumina iScan™ Readers, which use a laser to excite the fluorophore of the single-base extension product on the beads. The scanner records high-resolution images of the light emitted from the fluorophores. All plates and chips are barcoded and tracked with an internally derived laboratory information management system. The data from these images are analyzed to determine SNP genotypes using Illumina's BeadStudio. To support this process, Biomek F/X, three Tecan Freedom Evos, and two Tecan Genesis Workstation 150s can be used to automate all liquid handling steps throughout the sample and chip prep process.

Illumina BeadArray:

The Illumina Bead Lab system is a multiplexed array-based format. Illumina's BeadArray Technology is based on 3-micron silica beads that self-assemble in microwells on either of two substrates: fiber optic bundles or planar silica slides. When randomly assembled on one of these two substrates, the beads have a uniform spacing of −5.7 microns. Each bead is covered with hundreds of thousands of copies of a specific oligonucleotide that act as the capture sequences in one of Illumina's assays. BeadArray technology is utilized in Illumina's iScan System.

Sequenom:

During pre-PCR, either of two Packard Multiprobes is used to pool oligonucleotides, and a Tomtec Quadra 384 is used to transfer DNA. A Cartesian nanodispenser is used for small-volume transfer in pre-PCR, and another in post-PCR. Beckman Multimeks, equipped with either a 96-tip head or a 384-tip head, are used for more substantial liquid handling of mixes. Two Sequenom pin-tool are used to dispense nanoliter volumes of analytes onto target chips for detection by mass spectrometry. Sequenom Compact mass spectrometers can be used for genotype detection.

In some embodiments, methods provided herein comprise analyzing genomic DNA using a nucleic acid sequencing assay. Methods of genome sequencing are known in the art. Examples of genome sequencing methods and commercially available tools are described below.

Illumina Sequencing:

89 GAIIx Sequencers are used for sequencing of samples. Library construction is supported with 6 Agilent Bravo plate-based automation, Stratagene MX3005p qPCR machines, Matrix 2-D barcode scanners on all automation decks and 2 Multimek Automated Pipettors for library normalization.

454 Sequencing:

Roche® 454 FLX-Titanium instruments are used for sequencing of samples. Library construction capacity is supported by Agilent Bravo automation deck, Biomek FX and Janus PCR normalization.

SOLiD Sequencing:

SOLiD v3.0 instruments are used for sequencing of samples. Sequencing set-up is supported by a Stratagene MX3005p qPCR machine and a Beckman SC Quanter for bead counting.

ABI Prism® 3730 XL Sequencing:

ABI Prism® 3730 XL machines are used for sequencing samples. Automated Sequencing reaction set-up is supported by 2 Multimek Automated Pipettors and 2 Deerac Fluidics—Equator systems. PCR is performed on 60 Thermo-Hybaid 384-well systems.

Ion Torrent:

Ion PGM™ or Ion Proton™ machines are used for sequencing samples. Ion library kits (Invitrogen) can be used to prepare samples for sequencing.

Other Technologies:

Examples of other commercially available platforms include Helicos Heliscope Single-Molecule Sequencer, Polonator G.007, and Raindance RDT 1000 Rainstorm.

### Expression Level Analysis

The invention contemplates that elevated risk of developing MCC is associated with an altered expression pattern of a gene located at, within, or near a risk haplotype, such as a gene located in Table 3. The invention therefore contemplates methods that involve measuring the mRNA or protein levels for these genes and comparing such levels to control levels, including for example predetermined thresholds.

In some embodiments, a method described herein comprises measuring the level of an alternative splice variant mRNA of GNAI2. In some embodiments, the alternative splice variant mRNA is an mRNA excluding exon 3. In some embodiments, an increased level of the alternative splice variant identifies a subject as a subject (a) at elevated risk of developing a MCC or (b) having an undiagnosed MCC.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays.

Expression profiles of cells in a biological sample (e.g., blood or a tumor) can be carried out using an oligonucleotide microarray analysis. As an example, this analysis may be carried out using a commercially available oligonucleotide microarray or a custom designed oligonucleotide microarray comprising oligonucleotides for all or a subset of the transcripts described herein. The microarray may comprise any number of the transcripts, as the invention contemplates that elevated risk may be determined based on the analysis of single differentially expressed transcripts or a combination of differentially expressed transcripts. The transcripts may be those that are up-regulated in tumors carrying a germ-line risk marker (compared to a tumor that does not carry the germ-line risk marker), or those that are down-regulated in tumors carrying a germ-line risk marker (compared to a tumor that does not carry the germ-line risk marker), or a combination of these. The number of transcripts measured using the microarray therefore may be 1, 2, 3, 4, 5, 6, 7, 8, 9, or more transcripts encoded by a gene in Table 3. It is to be understood that such arrays may however also comprise positive and/or negative control transcripts such as housekeeping genes that can be used to determine if the array has been degraded and/or if the sample has been degraded or contaminated. The art is familiar with the construction of oligonucleotide arrays.

Commercially available gene expression systems include Affymetrix GeneChip microarrays as well as all of Illumina standard expression arrays, including two GeneChip 450 Fluidics Stations and a GeneChip 3000 Scanner, Affymetrix High-Throughput Array (HTA) System composed of a GeneStation liquid handling robot and a GeneChip HT Scanner providing automated sample preparation, hybridization, and scanning for 96-well Affymetrix PEGarrays. These systems can be used in the cases of small or potentially degraded RNA samples. The invention also contemplates analyzing expression levels from fixed samples (as compared to freshly isolated samples). The fixed samples include formalin-fixed and/or paraffin-embedded samples. Such samples may be analyzed using the whole genome Illumina DASL assay. High-throughput gene expression profile analysis can also be achieved using bead-based solutions, such as Luminex systems.

Other mRNA detection and quantitation methods include multiplex detection assays known in the art, e.g., xMAP® bead capture and detection (Luminex Corp., Austin, Tex.).

Another exemplary method is a quantitative RT-PCR assay which may be carried out as follows: mRNA is extracted from cells in a biological sample (e.g., blood or a tumor) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. Probes may be designed using the sequences or sequence identifiers listed in Table 3. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Assays

The art is familiar with various methods for measuring protein levels. Protein levels may be measured using protein-based assays such as but not limited to immunoassays, Western blots, Western immunoblotting, multiplex bead-based assays, and assays involving aptamers (such as SOMAmer™ technology) and related affinity agents.

A brief description of an exemplary immunoassay is provided here. A biological sample is applied to a substrate having bound to its surface protein-specific binding partners (i.e., immobilized protein-specific binding partners). The protein-specific binding partner (which may be referred to as a “capture ligand” because it functions to capture and immobilize the protein on the substrate) may be an antibody or an antigen-binding antibody fragment such as Fab, F(ab)2, Fv, single chain antibody, Fab and sFab fragment, F(ab′)₂, Fd fragments, scFv, and dAb fragments, although it is not so limited. Other binding partners are described herein. Protein present in the biological sample bind to the capture ligands, and the substrate is washed to remove unbound material. The substrate is then exposed to soluble protein-specific binding partners (which may be identical to the binding partners used to immobilize the protein). The soluble protein-specific binding partners are allowed to bind to their respective proteins immobilized on the substrate, and then unbound material is washed away. The substrate is then exposed to a detectable binding partner of the soluble protein-specific binding partner. In one embodiment, the soluble protein-specific binding partner is an antibody having some or all of its Fc domain. Its detectable binding partner may be an anti-Fc domain antibody. As will be appreciated by those in the art, if more than one protein is being detected, the assay may be configured so that the soluble protein-specific binding partners are all antibodies of the same isotype. In this way, a single detectable binding partner, such as an antibody specific for the common isotype, may be used to bind to all of the soluble protein-specific binding partners bound to the substrate.

It is to be understood that the substrate may comprise capture ligands for one or more proteins, including two or more, three or more, four or more, five or more, etc. up to and including all of the proteins encoded by the genes in Table 3 provided by the invention.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include protein-specific binding partners. Protein-specific binding partners can be generated using the sequences or sequence identifiers listed in Table 3. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target protein, e.g., through non-covalent bonding. For example, if the protein is a ligand, a binding partner may be a receptor for that ligand. In another example, if the protein is a receptor, a binding partner may be a ligand for that receptor. In yet another example, a binding partner may be a protein or peptide known to interact with a protein. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

Binding partners also include any molecule capable of demonstrating selective binding to any one of the target proteins disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

### Detectable Labels

Detectable binding partners may be directly or indirectly detectable. A directly detectable binding partner may be labeled with a detectable label such as a fluorophore. An indirectly detectable binding partner may be labeled with a moiety that acts upon (e.g., an enzyme or a catalytic domain) or a moiety that is acted upon (e.g., a substrate) by another moiety in order to generate a detectable signal. Exemplary detectable labels include, e.g., enzymes, radioisotopes, haptens, biotin, and fluorescent, luminescent and chromogenic substances. These various methods and moieties for detectable labeling are known in the art.

### Devices and Kits

Any of the methods provided herein can be performed on a device, e.g., an array. Suitable arrays are described herein and known in the art. Accordingly, a device, e.g., an array, for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 5, up to 10, up to 15, up to 20, up to 25, up to 30, up to 35, up to 40, up to 45, up to 50, up to 75 or up to 100 germ-line risk markers) described herein is also contemplated.

Reagents for use in any of the methods provided herein can be in the form of a kit. Accordingly, a kit for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 5, up to 10, up to 15, up to 20, up to 25, up to 30, up to 35, up to 40, up to 45, up to 50, up to 75 or up to 100 germ-line risk markers) described herein is also contemplated. In some embodiments, the kit comprises reagents for detecting any of the germ-line risk markers described herein, e.g., reagents for use in a method described herein. Suitable reagents are described herein and art known in the art.

### Controls

Some of the methods provided herein involve measuring a level or determining the identity of a germ-line risk marker in a biological sample and then comparing that level or identity to a control in order to identify a subject having an elevated risk of developing a MCC.

The control may be a control level or identity that is a level or identity of the same germ-line risk marker in a control tissue, control subject, or a population of control subjects.

The control may be (or may be derived from) a normal subject (or normal subjects). A normal subject, as used herein, refers to a subject that is healthy. The control population may be a population of normal subjects.

In other instances, the control may be (or may be derived from) a subject (a) having a similar cancer to that of the subject being tested and (b) who is negative for the germ-line risk marker.

It is to be understood that the methods provided herein do not require that a control level or identity be measured every time a subject is tested. Rather, it is contemplated that control levels or identities of germ-line risk markers are obtained and recorded and that any test level is compared to such a pre-determined level or identity (or threshold).

In some embodiments, a control is a non-risk nucleotide of a SNP, e.g., a non-risk nucleotide in Table 1A or 2. In some embodiments, a control is a non-risk nucleotide of a SNP, e.g., a non-risk nucleotide in Table 1B.

### Samples

The methods provided herein detect and optionally measure (and thus analyze) levels or particular germ-line risk markers in biological samples. Biological samples, as used herein, refer to samples taken or obtained from a subject. These biological samples may be tissue samples or they may be fluid samples (e.g., bodily fluid). Examples of biological fluid samples are whole blood, plasma, serum, urine, sputum, phlegm, saliva, tears, and other bodily fluids. In some embodiments, the biological sample is a whole blood or saliva sample. In some embodiments, the biological sample is a tumor, a fragment of a tumor, or a tumor cell(s). In some embodiments, the biological sample is a skin sample or skin biopsy.

In some embodiments, the biological sample may comprise a polynucleotide (e.g., genomic DNA or mRNA) derived from a tissue sample or fluid sample of the subject. In some embodiments, the biological sample may comprise a polypeptide (e.g., a protein) derived from a tissue sample or fluid sample of the subject. In some embodiments, the biological sample may be manipulated to extract a polynucleotide or polypeptide. In some embodiments, the biological sample may be manipulated to amplify a polynucleotide sample. Methods for extraction and amplification are well known in the art.

### Subjects

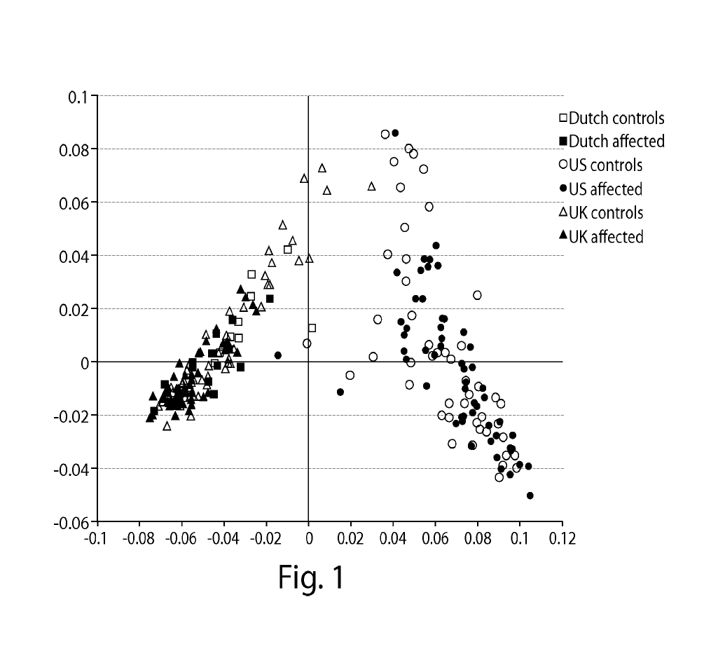

Methods of the invention are intended for canine subjects. In some embodiments, canine subjects include, for example, those with a higher incidence of MCC as determined by breed. For example the canine subject may be a Golden Retriever (GR), a Labrador Retriever, a Chinese Shar-Pei, a Boxer, a Pug, or a Boston Terrier, or a descendant of a Golden Retriever, a Labrador Retriever, a Chinese Shar-Pei, a Boxer, a Pug, or a Boston Terrier. In some embodiments, the canine subject is Golden Retriever or a descendant of a Golden Retriever. As used herein, a “descendant” includes any blood relative in the line of descent, e.g., first generation, second generation, third generation, fourth generation, etc., of a canine subject. Such a descendant may be a pure-bred canine subject, e.g., a descendant of two Golden Retriever parents, or a mixed-breed canine subject, e.g., a descendant of both a pure-bred Golden Retriever and a non-Golden Retriever. Breed can be determined, e.g., using commercially available genetic tests (see, e.g., Wisdom Panel). In some embodiments, a canine subject is of European or American descent. In some embodiments, a canine subject is of European descent. In some embodiments, a canine subject is of American descent. American and European descent can be determined by genotyping (e.g., using the Illumina 170K canine HD SNP array) as the dogs from the two continents will separate in a simple principal component analysis (see FIG. 1). Additionally or alternatively, physical features may be used to distinguish canine subjects of European or American descent as breed standards for each continent vary. For example, the American kennel club does not recognize pale cream-colored Golden Retrievers, but pale cream-colored Golden Retrievers are recognized by the British kennel club.

Methods of the invention may be used in a variety of other subjects including but not limited to human subjects.

### Computational Analysis

Methods of computation analysis of genomic and expression data are known in the art. Examples of available computational programs are: Genome Analysis Toolkit (GATK, Broad Institute, Cambridge, Mass.), Expressionist Refiner module (Genedata AG, Basel, Switzerland), GeneChip—Robust Multichip Averaging (CG-RMA) algorithm, PLINK (Purcell et al, 2007), GCTA (Yang et al, 2011), the EIGENSTRAT method (Price et al 2006), EMMAX (Kang et al, 2010). In some embodiments, methods described herein include a step comprising computational analysis.

### Breeding Programs

Other aspects of the invention relate to use of the diagnostic methods in connection with a breeding program. A breeding program is a planned, intentional breeding of a group of animals to reduce detrimental or undesirable traits and/or increase beneficial or desirable traits in offspring of the animals. Thus, a subject identified using the methods described herein as not having a germ-line risk marker of the invention may be included in a breeding program to reduce the risk of developing MCC in the offspring of said subject. Alternatively, a subject identified using the methods described herein as having a germ-line risk marker of the invention may be excluded from a breeding program. In some embodiments, methods of the invention comprise exclusion of a subject identified as being at elevated risk of developing MCC in a breeding program or inclusion of a subject identified as not being at elevated risk of developing MCC in a breeding program.

### Treatment

Other aspects of the invention relate to diagnostic or prognostic methods that comprise a treatment step (also referred to as “theranostic” methods due to the inclusion of the treatment step). Any treatment for MCC is contemplated. In some embodiments, treatment comprises one or more of surgery, chemotherapy, and radiation. Examples of chemotherapy for treatment of MCCs include, but are not limited to, prednisone, Toceranib, Masitinib, vinblastine, and Lomustine. Surgery may be combined with the use of antihistamines (e.g. diphenhydramine) and/or H2 blockers (e.g., cimetidine) to protect a subject against histamine release from the tumor during surgical removal.

In some embodiments, a subject identified as being at elevated risk of developing MCC or having undiagnosed MCC is treated. In some embodiments, the method comprises selecting a subject for treatment on the basis of the presence of one or more germ-line risk markers as described herein. In some embodiments, the method comprises treating a subject with a MCC characterized by the presence of one or more germ-line risk markers as defined herein. As described herein, it was discovered that hyaluronidase genes are significantly associated with MCC in canine subjects. Hyaluronidase enzymes degrade the glucosaminoglycan hyaluronic acid (HA). HA is a major component of the extracellular matrix and cellular microenvironment. Without wishing to be bound by theory, alteration of HA degradation may lead to changes in the extracellular microenvironment that may lead to MCC.

The invention contemplates blockade of HA signaling (e.g., by degrading HA, by degrading a receptor for HA, such as CD44, or by blocking the interaction of HA and a receptor for HA, such as CD44) may prevent or treat MCC. Accordingly, methods for treatment of subjects with MCC are provided. The subject may or may not have one or more of the germ-line risk markers as defined herein. In some embodiments, treatment comprises administering a CD44 inhibitor and/or an HA inhibitor to a subject having MCC. CD44 and/or HA can be inhibited using any method known in the art. Inhibition of activity and/or production of CD44 and/or HA may be achieved, e.g., by using nucleic acids such as DNA and RNA aptamers, antisense oligonucleotides, siRNA and shRNA, small peptides, antibodies or antibody fragments, and small molecules such as small chemical compounds. Such inhibitors may be designed, e.g., using the sequence of CD44 (ENSCAFG00000006889 or ENSG00000026508).

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin). Dosage will depend on the subject and the route of administration. Dosage can be determined by the skilled artisan.

### EXAMPLES

### Example 1

### Methods

### Samples

All blood samples were collected from pet dogs after owner consent according to ethical approval protocols of the collection institutions. A total of 106 Golden Retriever samples were collected in the United States (58 cases and 48 controls), 113 in the United Kingdom (53 cases and 60 controls) and 33 in the Netherlands (18 cases and 15 controls). Genomic DNA was extracted from whole blood or buccal swabs using QIAamp DNA Blood Midi Kit (QIAGEN), Nucleon® Genomic DNA Extraction Kit (Tepnel Life Sciences), phenol-chloroform extraction [ref. 33] or salt extraction [ref. 34]. All cases were diagnosed as mast cell tumours by cytology or histopathology. The control dogs were healthy without tumor diagnosis and over 7 years old. Only one dog was included from each litter to reduce the amount of relatedness in the sample set.

### Genome-Wide Association (GWAS) Mapping

The Illumina 170K canine HD SNP arrays were used for genotyping of approximately 174,000 SNPs with a mean genomics distance of 13 Kb [ref. 35]. The genotyping was performed at the Centre National de Genotypage, France, Broad Institute, USA, and Geneseek (Neogen), USA. The American and European Golden Retriever cohorts were analysed both separately and as a joint dataset. Data quality control was performed using the software package PLINK [ref. 36], removing SNPs and individuals with a call rate below 90%. SNPs with a minor allele frequency below 0.1% were also removed from further association analysis. Population stratification was estimated and visualized in multi-dimensional scaling plots (MDS) using PLINK (FIG. 1) to detect outliers and subgroups in the dataset after pruning out SNPs in high linkage disequilibrium (r²>0.95). Due to the cryptic relatedness in dog breeds, the level of relatedness between individuals was calculated using the GCTA software [ref. 37], and a 0.25 cut-off was used to remove highly related dogs (corresponding to half-sibs) while maximising the number of individuals remaining in the dataset. The genome was screened for regions associated with mast cell cancer (MCC) using a case-control genome-wide association analysis. The EMMAX software was used to calculate association p-values corrected for stratification and cryptic relatedness using mixed model statistics. The two primary eigenvectors calculated using the GCTA software [ref. 37] were used as covariates in the analysis to adjust for stratification. The LD pruned SNP set was used for the estimations of MDS, relatedness and eigenvectors in GCTA and relationship matrix in EMMAX, whereas the full QC filtered SNP set was used for the association testing. Quantile-quantile plots were created in R to assess possible genomic inflation and to establish suggestive significance levels [ref. 38]. Permutation testing was performed in GenABEL using mixed model statistics, two eigenvector covariates and 10,000 permutations [ref. 39].

Pair-wise linkage disequilibrium between markers was used to evaluate the size of candidate regions and whether the association peaks were independent. LD r² calculations were performed using the Haploview [ref. 40] and PLINK software packages [ref. 36]. Haplotype analysis was performed using Haploview [ref. 40] to identify haplotype structures in the candidate regions.

Gene annotations were extracted from ENSEMBL genome browser.

### Results

A case-control genome-wide association study (GWAS) of 252 Golden Retrievers (GR) was conducted to find candidate regions associated with mast cell cancer (MCC). After quality control and removal of related individuals, the GWAS included a total of 113 cases and 102 controls with low levels of relatedness (<0.25 relatedness coefficient) and high genotype call rates (>90%).

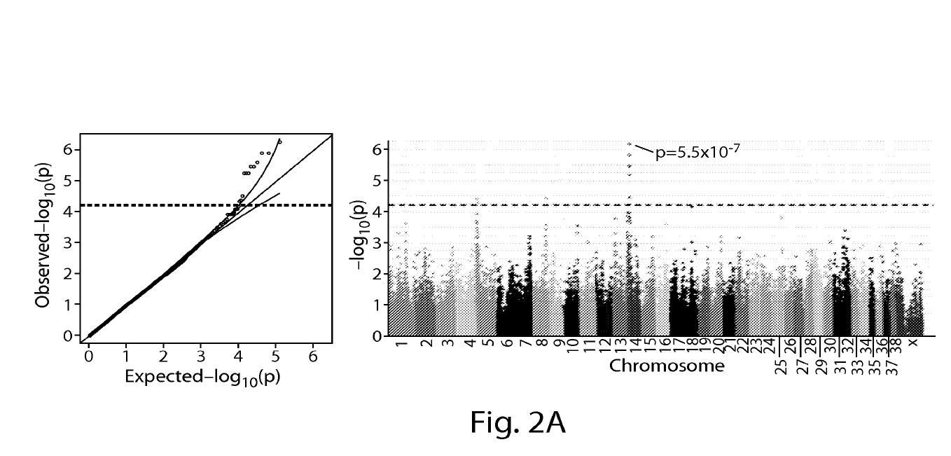

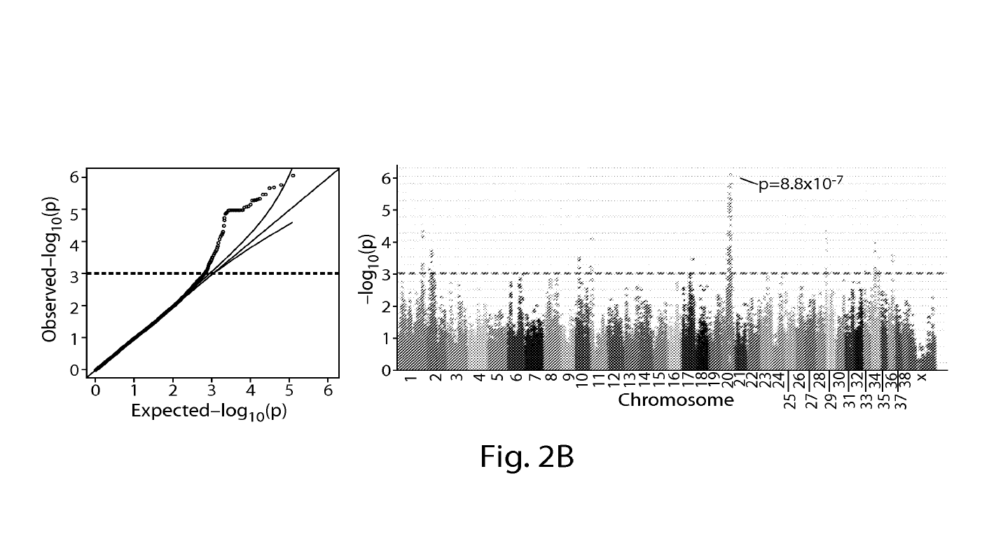

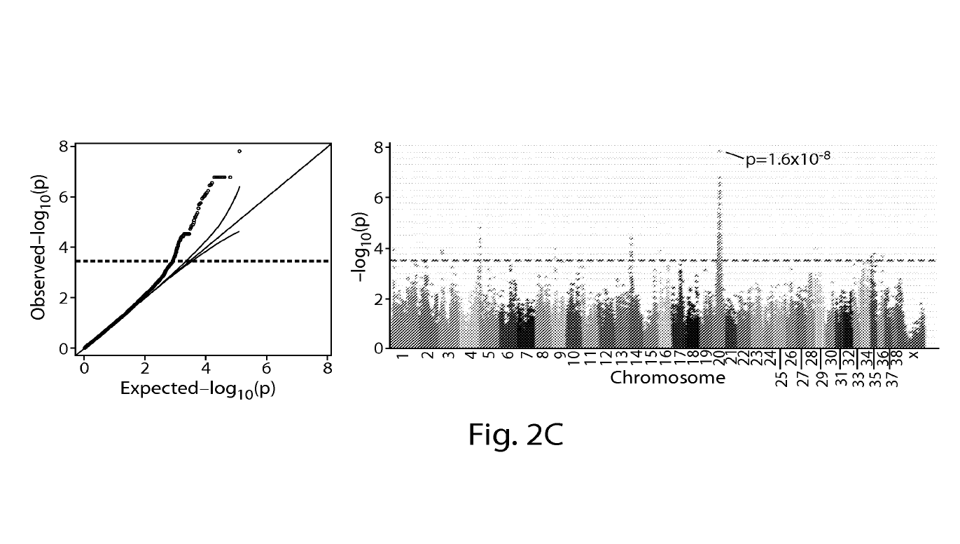

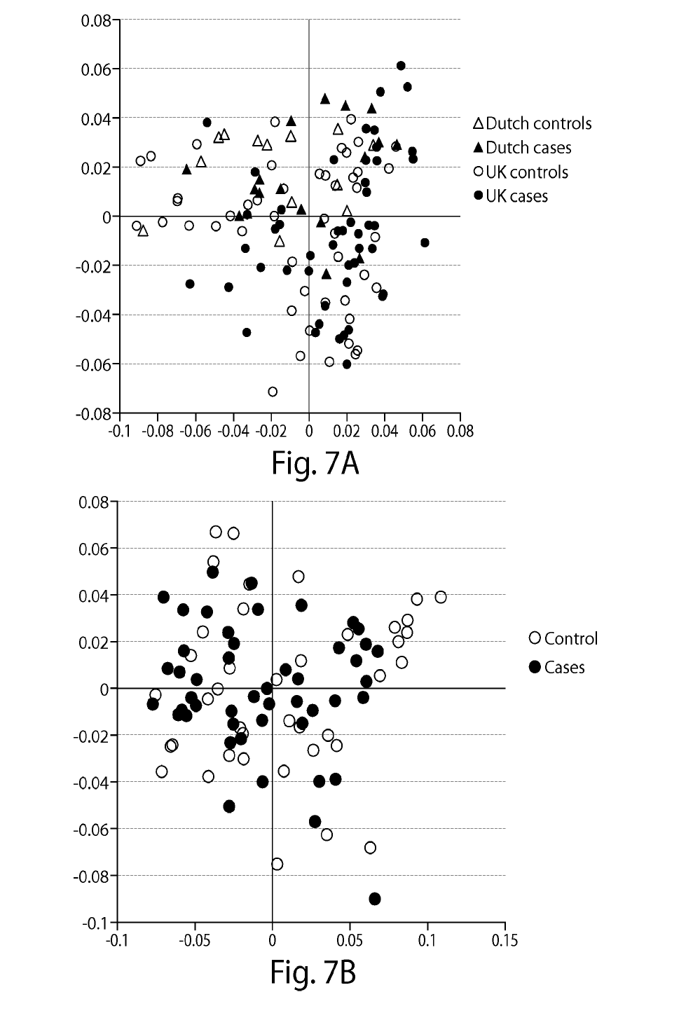

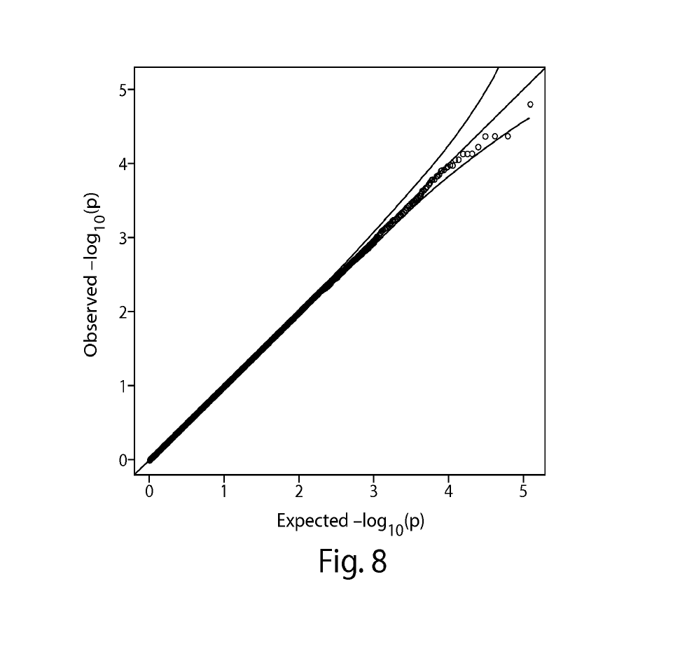

The multidimensional scaling plot (MDS) shows that the American and European GRs form two distinct clusters, indicating genetic dissimilarities between the populations on the different continents (FIG. 1). This implies that the MCT predisposition could have different genetic causes in the two populations. The two cohorts were analysed first separately, and then together. MDS plots for the two groups separately indicate no outliers or substantial stratification within the American and European cohorts respectively (FIG. 7). No residual genomic inflation was detected after corrections, as is noted from the QQ plots and genomic inflation factors (X=1.00 and 1.00, respectively, FIG. 2). The full cohort analysis resulted in minor residual genomic inflation after corrections, X=1.05. The elevated X is due to high LD in the top associated locus, giving association signal over several Mb, which is evident from the QQ plot after removing all SNPs in this region and rerunning the analysis (X=0.97, FIG. 8).

The Manhattan plots for the two different populations (FIGS. 2A and B) show one major associated locus for each population. The two peaks are however not overlapping but on different chromosomes (i.e., 14 and 20) confirming that different genetic risk factors are influencing the two populations of GR dogs.

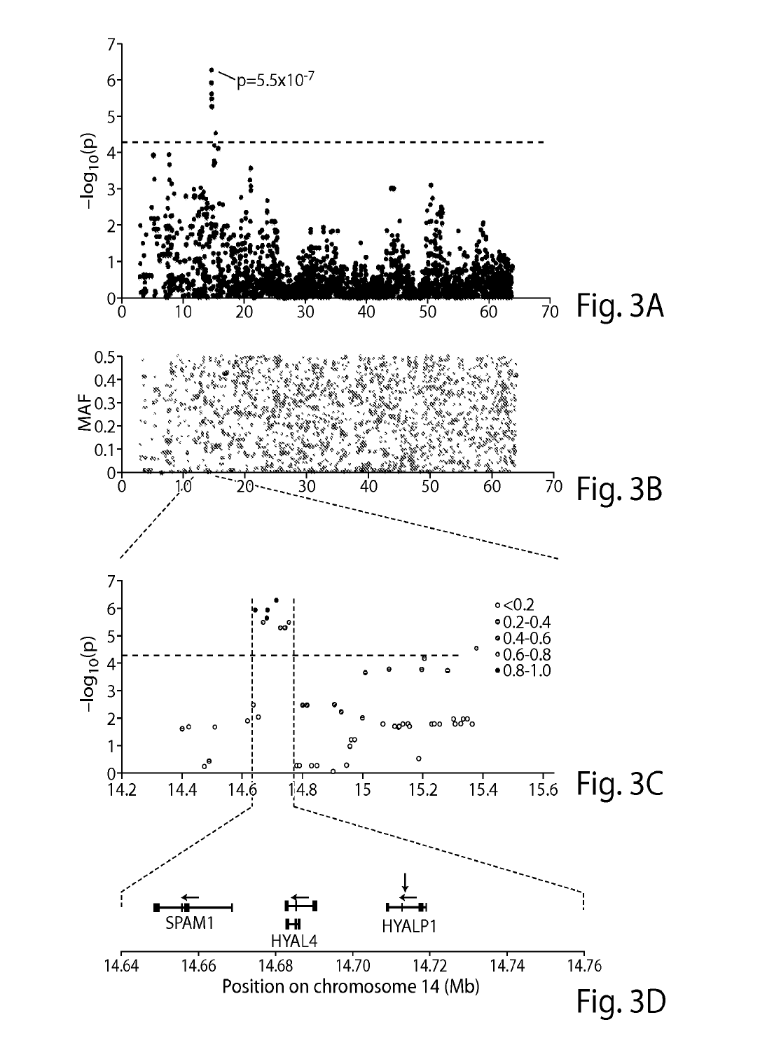

The American GR association analysis resulted in three nominally associated regions (−log p>4.2, based on a deviation in the QQ plot), on chromosome 5 (1 significant SNP), chromosome 8 (1 significant SNP) and chromosome 14 (10 significant SNPs) (FIG. 2A). The strongest association is on chromosome 14 (CanFam 2.0 Chr14:14.64-15.38 Mb) with the best SNP at p=5.5×10⁻⁷, pₚₑᵣₘ=0.065 (Chr14:14,714,009 bp) conferring a substantial risk (OR=0.13, FIG. 3). The risk allele frequency is 89% in cases and 50% in control American GRs. The top five SNPs are presented in Table 5A and B, and all significant SNPs are listed in Table 1A. All of the significant SNPs on chromosome 14 show high LD with the top SNP (FIG. 3C). Nine SNPs form a risk haplotype spanning 111 Kb (14.64-14.76 Mb) containing only three genes; SPAM1, HYAL4 and HYALP1. Notably, the genes are all hyaluronidase enzymes. The top SNP is located within the 2nd intron of HYALP1.

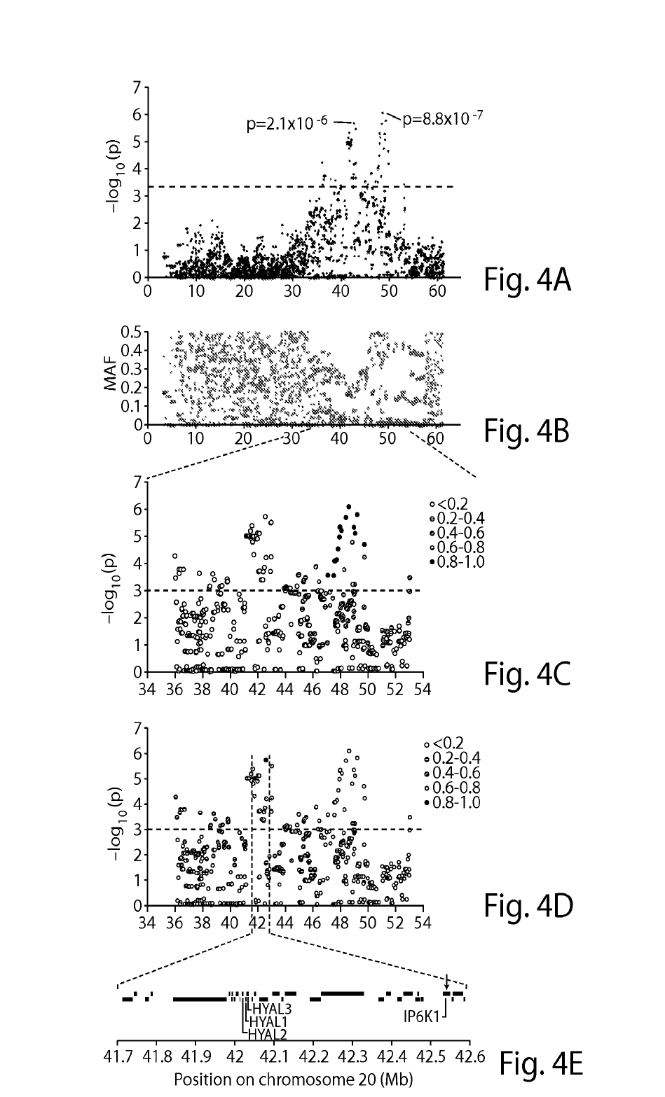

In the European population, chromosome 20 has the strongest association, while ten chromosomes show nominal significance (−log p>3, based on the QQ-plot, FIG. 2B). On chromosome 20, 135 SNPs spanning 17 Mb show nominal significance. They form two major loci at 42 Mb (41.70-42.59 Mb, best SNP p=2.1×10⁻⁶, pₚₑᵣₘ=0.068, OR=0.16, chr20:42,547,825 bp) and 49 Mb (47.06-49.70 Mb, best SNP p=8.8×10⁻⁷, pₚₑᵣₘ=0.032, OR=4.1, chr20:48,599,799 bp). Analysis of the linkage disequilibrium in this area shows that the top SNPs in each region are in high LD with nearby SNPs but low LD (r²<0.2) with SNPs in the other peak (FIG. 4). The risk allele frequency for the 42 Mb SNP is high, with an allele frequency of 91% in cases (n=65) and 66% in controls (n=62). The haplotype at 49 Mb is however less common, with a frequency of 65% in cases and 31% in controls, and the discrepancy in allele frequencies further supports that the associated loci are independent and could harbour separate risk factors for canine MCC. The differences in haplotype allele frequencies are also evident from the minor allele frequency plot (FIG. 4B). The minor allele frequency is reduced around 42 Mb, indicating a reduction in genetic diversity, possibly due to selection in that region. The large 17.0 Mb candidate region contains nearly 500 genes and corresponds to 3p21 in the human genome. The top SNP at 48 Mb falls between the MYO9B and HAUS8 genes and interestingly, there is a cluster of hyaluronidase genes (HYAL1, HYAL2 and HYAL3) positioned within the association peak at 42 Mb.

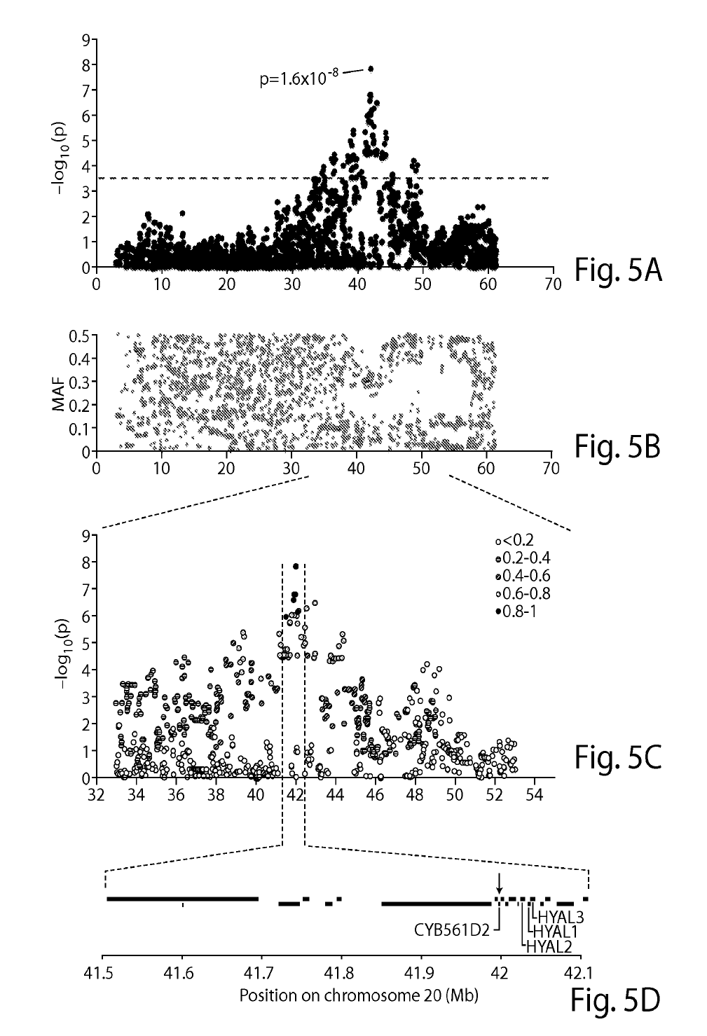

As expected, the full cohort GWAS results shows partial overlap with the American and European subsets (FIG. 2C). Interestingly, the peak at chr20:42 Mb is enhanced (best SNP p=1.6×10⁻⁸, pₚₑᵣₘ=0.024, CanFam 2.0 Chr20:42,004,062 bp, Table 5). The nominal significance threshold was set to −log p>3.5 to control for the slightly elevated genomic inflation stemming from one large association peak (X=1.05). 153 SNPs were nominally significant (Table 1A) and, out of these, 119 are positioned at the chr20:42 Mb locus (±10 Mb of top SNP). Nine top SNPs form a haplotype at 41.51-42.12 Mb (FIG. 5). The haplotype covers 18 genes, including the HYAL cluster containing HYAL1, HYAL2 and HYAL3. The top SNP at 42,004,062 by is positioned within the CYB561D2 gene 25 Kb from the HYAL genes. The top haplotypes identified in the European and full cohort overlap at 41.70-42.12 Mb, restricting the candidate interval to 17 genes, including the HYAL cluster.

TABLE 5A
Top 5 associated SNPs identified in the American, European and combined cohorts.

| Cohort | SNP ID | CHR | POSITION | Alleles | P_{US} | P_{EU} | P_{Comb} | Pₚₑᵣₘ | OR | MAF_{A} | MAF_{U} |
|---|---|---|---|---|---|---|---|---|---|---|---|
| American | BICF2G630521558 | 14 | 14644897 | T/C | 1.2E−06 | 0.179 | 0.002 | 0.142 | 0.14 | 0.11 | 0.49 |
| | BICF2G630521606 | 14 | 14682089 | C/T | 2.5E−06 | 0.170 | 0.002 | 0.270 | 0.15 | 0.13 | 0.49 |
| | BICF2G630521619 | 14 | 14685543 | T/C | 1.2E−06 | 0.170 | 0.002 | 0.142 | 0.14 | 0.11 | 0.49 |
| | BICF2G630521572 | 14 | 14670361 | C/T | 3.4E−06 | 0.066 | 4.3E−05 | 0.420 | 0.16 | 0.20 | 0.60 |
| | BICF2P867665 | 14 | 14714009 | T/G | 5.5E−07 | 0.223 | 0.001 | 0.065 | 0.13 | 0.11 | 0.50 |
| European | BICF2S22934685 | 20 | 42547825 | T/C | 0.781 | 2.1E−06 | 5.7E−07 | 0.068 | 0.16 | 0.08 | 0.36 |
| | BICF2P1444805 | 20 | 42957449 | G/A | 0.078 | 3.4E−06 | 3.5E−07 | 0.117 | 0.15 | 0.06 | 0.30 |
| | BICF2P299292 | 20 | 48377580 | A/C | 0.436 | 2.2E−06 | 1.1E−04 | 0.081 | 3.98 | 0.65 | 0.31 |
| | BICF2P301921 | 20 | 48599799 | A/C | 0.347 | 8.8E−07 | 6.4E−05 | 0.032 | 4.13 | 0.65 | 0.31 |
| | BICF2P623297 | 20 | 49201505 | G/A | 0.386 | 1.7E−06 | 9.5E−05 | 0.056 | 4.18 | 0.63 | 0.29 |
| Combined | BICF2P304809 | 20 | 41924733 | T/C | 0.015 | 1.3E−05 | 1.7E−07 | 0.122 | 0.37 | 0.23 | 0.45 |
| | BICF2P1310301 | 20 | 41927031 | A/G | 0.015 | 1.3E−05 | 1.7E−07 | 0.122 | 0.37 | 0.23 | 0.45 |
| | BICF2P1310305 | 20 | 41930509 | A/G | 0.015 | 1.3E−05 | 1.7E−07 | 0.122 | 0.37 | 0.23 | 0.45 |
| | BICF2P1231294 | 20 | 41951828 | C/T | 0.015 | 1.3E−05 | 1.7E−07 | 0.122 | 0.37 | 0.23 | 0.45 |
| | BICF2P1185290 | 20 | 42004062 | T/C | 0.007 | 8.1E−06 | 1.6E-08 | 0.024 | 0.34 | 0.22 | 0.45 |

CHR,chromosome; Alleles, minor/major allele; P_{US}, P value of the US cohort; P_{EU}, P value of the European cohort; P_{Comb}, P value of combined, full cohort; Pₚₑᵣₘ, permuted P value for the population where top 5 significance was established; OR, Odds ratio for minor allele in the population where top 5 significance was established; MAF_{A}, minor allele frequency for affected in the population where top 5 significance was established; MAF_{U}, minor allele frequency for unaffected in the population where top 5 significance was established. Nominal significance is indicated in bold.

TABLE 5B
Top 5 associated SNPs identified in the American,
European and combined cohorts.

| | | | | | | Refer- |
|---|---|---|---|---|---|---|
| Cohort | SNP ID | CHR | POSITION | Alleles | Risk | ence |
| American | BICF2G630521558 | 14 | 14644897 | T/C | C | C |
| | BICF2G630521606 | 14 | 14682089 | C/T | T | T |
| | BICF2G630521619 | 14 | 14685543 | T/C | C | C |
| | BICF2G630521572 | 14 | 14670361 | C/T | T | T |
| | BICF2P867665 | 14 | 14714009 | T/G | G | T |
| European | BICF2S22934685 | 20 | 42547825 | T/C | C | T |
| | BICF2P1444805 | 20 | 42957449 | G/A | A | G |
| | BICF2P299292 | 20 | 48377580 | A/C | A | A |
| | BICF2P301921 | 20 | 48599799 | A/C | A | C |
| | BICF2P623297 | 20 | 49201505 | G/A | G | A |
| Combined | BICF2P304809 | 20 | 41924733 | T/C | C | C |
| | BICF2P1310301 | 20 | 41927031 | A/G | G | G |
| | BICF2P1310305 | 20 | 41930509 | A/G | G | G |
| | BICF2P1231294 | 20 | 41951828 | C/T | T | T |
| | BICF2P1185290 | 20 | 42004062 | T/C | C | C |

CHR, chromosome;
Alleles, minor/major allele;
Risk, risk allele;
Reference = nucleotide identity in Boxer reference genome

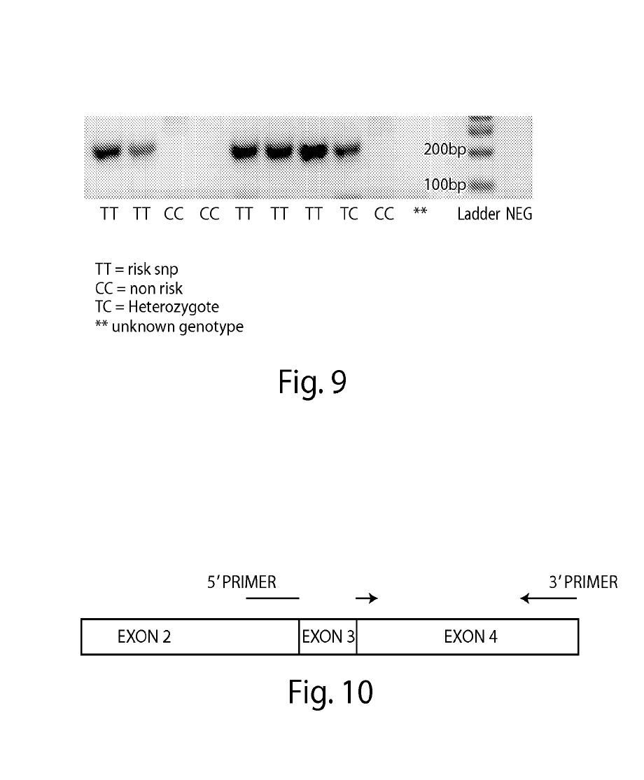

An additional top SNP (CanFam 2.0, Chr20:4,208,0147 bp, P value (EU cohort)=1.09 E¹⁵, P value (US cohort)=0.0023) was identified by sequencing of individuals with the risk haplotype and fine mapping. This SNP is located as the last basepair in the third exon of the GNAI2 gene. This location converts the splice site at the exon junction from a strong to a relative weak splice site. This results in alternative splicing of the GNAI2 mRNA by skipping exon 3. The alternative splice form can be identified by splice specific primers. FIG. 9 shows the results of PCR products formed using splice specific primers (FIG. 10). Only samples carrying the risk genotype produce the alternative splice form. The allele frequencies for this SNP are shown in Table 6.

TABLE 6
Chr20: 4,208,0147 bp SNP allele frequencies in EU and US cohort

| | TOTAL | TT | TC | CC |
|---|---|---|---|---|
| EU cohort | | | | |
| Controls | 65 | 6 | 33 | 26 |
| Cases | 65 | 45 | 18 | 2 |
| US cohort | | | | |
| Controls | 152 | 1 | 3 | 148 |
| Cases | 99 | 0 | 10 | 89 |

T = risk allele,
C= non-risk allele

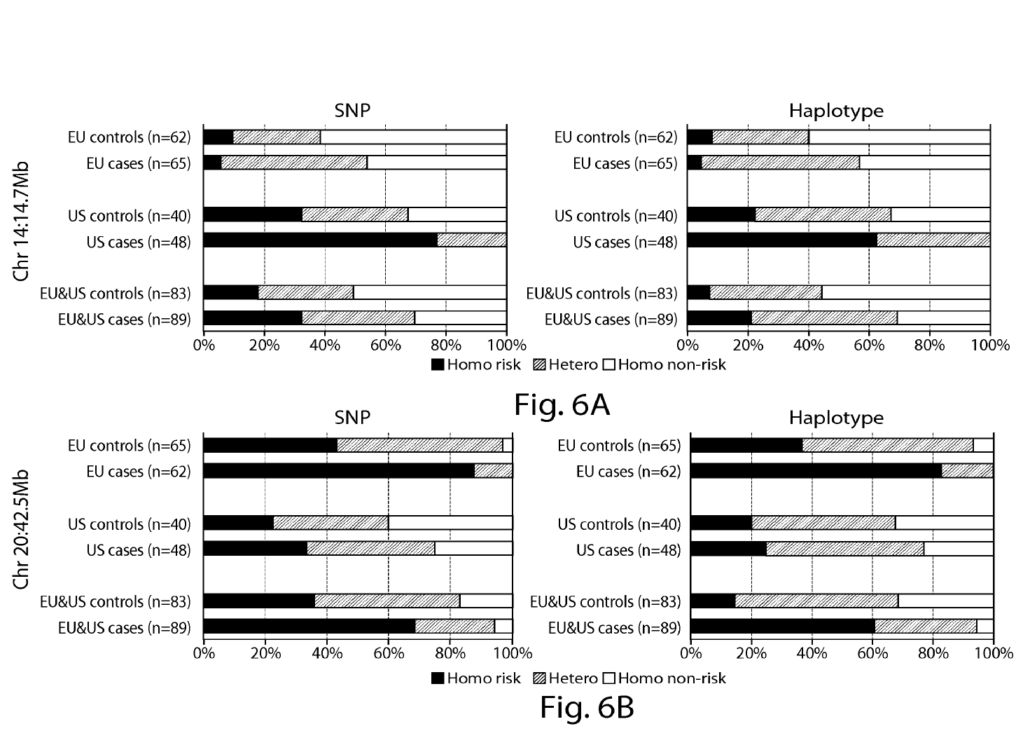

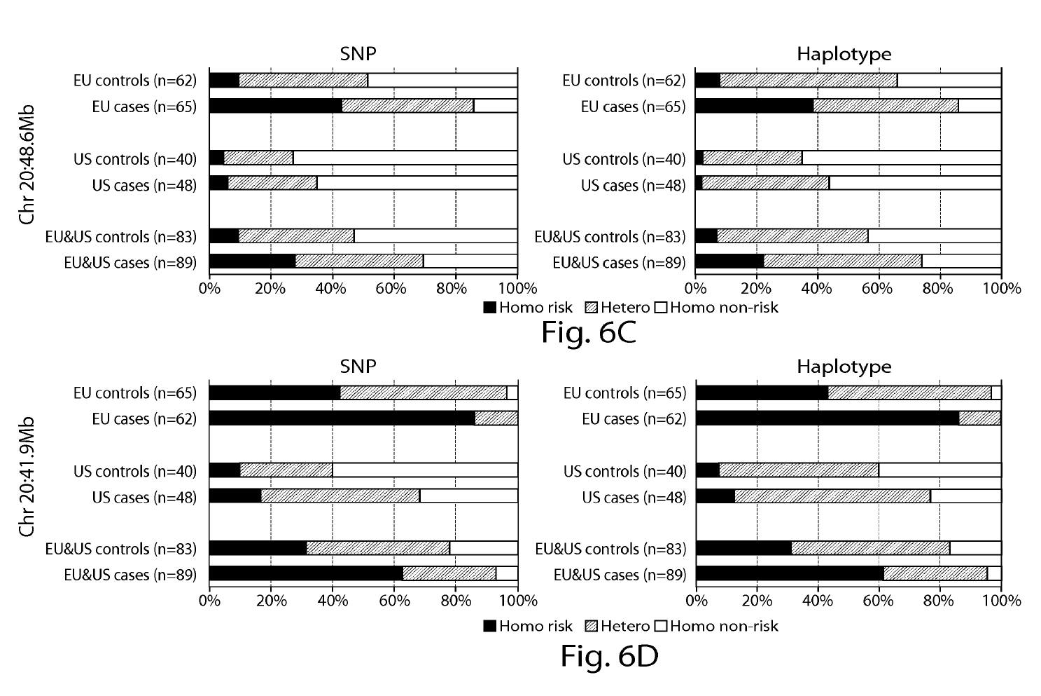

FIG. 6 shows the SNP and risk haplotype frequencies on chromosomes 14 and 20 in all cohorts. FIG. 6(a) shows the allele frequencies for both the top SNP and the haplotype on chromosome 14. For the top SNP on chromosome 14 (BICF2P867665) approximately 100% of the US case population was heterozygous or homozygous for the risk allele, while approximately 66% of the US control population was heterozygous or homozygous for the risk allele. For the same SNP (BICF2P867665) in the EU cohort, approximately 55% of the EU case population was heterozygous or homozygous for the risk allele, while approximately 40% of the EU control population was heterozygous or homozygous for the risk allele. For the same SNP (BICF2P867665) in the combined cohort, approximately 70% of the combined case population was heterozygous or homozygous for the risk allele, while approximately 50% of the combined control population was heterozygous or homozygous for the risk allele.

For the haplotype on chromosome 14 (14.64-14.76 Mb) approximately 100% of the US case population was heterozygous or homozygous for the risk haplotype, while approximately 66% of the US control population was heterozygous or homozygous for the risk haplotype. For the same haplotype on chromosome 14 (14.64-14.76 Mb) in the EU cohort, approximately 55% of the EU case population was heterozygous or homozygous for the risk haplotype, while approximately 40% of the EU control population was heterozygous or homozygous for the risk haplotype. For the same haplotype on chromosome 14 (14.64-14.76 Mb) in the combined cohort, approximately 70% of the combined case population was heterozygous or homozygous for the risk haplotype, while approximately 45% of the combined control population was heterozygous or homozygous for the risk haplotype.

FIG. 6(b) shows the allele frequencies for both the top SNP and the haplotype near Chr20:42.5 Mb. For the top SNP near Chr20:42.5 Mb (BICF2S22934685) approximately 75% of the US case population was heterozygous or homozygous for the risk allele, while approximately 60% of the US control population was heterozygous or homozygous for the risk allele. For the same SNP (BICF2S22934685) in the EU cohort, approximately 100% of the EU case population was heterozygous or homozygous for the risk allele, with approximately 85% being homozygous for the risk allele, while approximately 90% of the EU control population was heterozygous or homozygous for the risk allele, with approximately 45% being homozygous for the risk allele. For the same SNP (BICF2S22934685) in the combined cohort, approximately 90% of the combined case population was heterozygous or homozygous for the risk allele, with approximately 70% being homozygous for the risk allele, while approximately 80% of the combined control population was heterozygous or homozygous for the risk allele with approximately 35% being homozygous for the risk allele.

For the haplotype near Chr20:42.5 Mb (41.70-42.59 Mb) approximately 75% of the US case population was heterozygous or homozygous for the risk haplotype, while approximately 60% of the US control population was heterozygous or homozygous for the risk haplotype. For the same haplotype (41.70-42.59 Mb) in the EU cohort, approximately 100% of the EU case population was heterozygous or homozygous for the risk haplotype, with approximately 85% being homozygous for the risk haplotype, while approximately 90% of the EU control population was heterozygous or homozygous for the risk haplotype, with approximately 40% being homozygous for the risk haplotype. For the same haplotype (41.70-42.59 Mb) in the combined cohort, approximately 90% of the combined case population was heterozygous or homozygous for the risk haplotype, with approximately 60% being homozygous for the risk haplotype, while approximately 70% of the combined control population was heterozygous or homozygous for the risk haplotype, with approximately 15% being homozygous for the risk haplotype.

FIG. 6(c) shows the allele frequencies for both the top SNP and the haplotype near Chr20:48.6 Mb. For the top SNP near Chr20:48.6 Mb (BICF2P301921) approximately 40% of the US case population was heterozygous or homozygous for the risk allele, while approximately 30% of the US control population was heterozygous or homozygous for the risk allele. For the same SNP (BICF2P301921) in the EU cohort, approximately 90% of the EU case population was heterozygous or homozygous for the risk allele, while approximately 50% of the EU control population was heterozygous or homozygous for the risk allele. For the same SNP (BICF2P301921) in the combined cohort, approximately 70% of the combined case population was heterozygous or homozygous for the risk allele, while approximately 50% of the combined control population was heterozygous or homozygous for the risk allele.

For the haplotype near Chr20:48.6 Mb (47.06-49.70 Mb) approximately 45% of the US case population was heterozygous or homozygous for the risk haplotype, while approximately 35% of the US control population was heterozygous or homozygous for the risk haplotype. For the same haplotype (47.06-49.70 Mb) in the EU cohort, approximately 90% of the EU case population was heterozygous or homozygous for the risk haplotype, while approximately 65% of the EU control population was heterozygous or homozygous for the risk haplotype. For the same haplotype (47.06-49.70 Mb) in the combined cohort, approximately 75% of the combined case population was heterozygous or homozygous for the risk haplotype, while approximately 60% of the combined control population was heterozygous or homozygous for the risk haplotype.

FIG. 6(d) shows the allele frequencies for both the top SNP and the haplotype near Chr20:41.9 Mb. For the top SNP near Chr20:41.9 Mb (BICF2P1185290) approximately 70% of the US case population was heterozygous or homozygous for the risk allele, while approximately 40% of the US control population was heterozygous or homozygous for the risk allele. For the same SNP (BICF2P1185290) in the EU cohort, approximately 100% of the EU case population was heterozygous or homozygous for the risk allele, with approximately 90% being homozygous for the risk allele, while approximately 95% of the EU control population was heterozygous or homozygous for the risk allele, with approximately 40% being homozygous for the risk allele. For the same SNP (BICF2P1185290) in the combined cohort, approximately 90% of the combined case population was heterozygous or homozygous for the risk allele, with approximately 60% being homozygous for the risk allele, while approximately 75% of the combined control population was heterozygous or homozygous for the risk allele, with approximately 30% being homozygous for the risk allele.

For the haplotype on near Chr20:41.9 Mb (41.51-42.12 Mb) approximately 75% of the US case population was heterozygous or homozygous for the risk haplotype, while approximately 60% of the US control population was heterozygous or homozygous for the risk haplotype. For the same haplotype (41.51-42.12 Mb) in the EU cohort, approximately 100% of the EU case population was heterozygous or homozygous for the risk haplotype, with approximately 80% being homozygous for the risk haplotype, while approximately 95% of the EU control population was heterozygous or homozygous for the risk haplotype, with approximately 45% being homozygous for the risk haplotype. For the same haplotype (41.51-42.12 Mb) in the combined cohort, approximately 95% of the combined case population was heterozygous or homozygous for the risk haplotype, with approximately 60% being homozygous for the risk haplotype, while approximately 80% of the combined control population was heterozygous or homozygous for the risk haplotype, with approximately 30% being homozygous for the risk haplotype.

A listing of the allele frequencies for each SNP is provided in Table 7.

TABLE 7
SNP allele frequencies

| | | | | Allele | Allele | | Allele | Allele | |
|---|---|---|---|---|---|---|---|---|---|
| | | | | freq | freq | | freq | freq | |
| CHR | SNP | POSITION | A1 | affected | control | A2 | affected | control | REF |
| 14 | chr14: 14610095 | 14610095 | T | 0.1319 | 0.106 | A | 0.8681 | 0.894 | A |
| 14 | chr14: 14644897 | 14644897 | C | 0.5967 | 0.4925 | T | 0.4033 | 0.5075 | C |
| 14 | chr14: 14653880 | 14653880 | C | 0.39 | 0.3125 | T | 0.61 | 0.6875 | T |
| 14 | chr14: 14661891 | 14661891 | G | 0.36 | 0.295 | A | 0.54 | 0.705 | A |
| 14 | chr14: 14664532 | 14664532 | T | 0.37 | 0.2975 | C | 0.63 | 0.7025 | C |
| 14 | chr14: 14666424 | 14666424 | C | 0.4567 | 0.3518 | T | 0.5433 | 0.6482 | T |
| 14 | chr14: 14682089 | 14682089 | T | 0.5946 | 0.4974 | C | 0.4054 | 0.5026 | T |
| 14 | chr14: 14685543 | 14685543 | C | 0.6067 | 0.5025 | T | 0.3933 | 0.4975 | C |
| 14 | chr14: 14685602 | 14685602 | G | 0.6483 | 0.5309 | A | 0.3517 | 0.4691 | G |
| 14 | chr14: 14685771 | 14685771 | G | 0.6067 | 0.505 | T | 0.3933 | 0.495 | G |
| 14 | chr14: 14714009 | 14714009 | G | 0.5957 | 0.5208 | T | 0.4043 | 0.4792 | T |
| 14 | chr14: 14767603 | 14767603 | C | 0.37 | 0.2854 | T | 0.63 | 0.7146 | C |
| 14 | chr14: 14767966 | 14767966 | C | 0.37 | 0.2864 | T | 0.63 | 0.7136 | C |
| 14 | chr14: 14827179 | 14827179 | C | 0.5205 | 0.4492 | A | 0.4795 | 0.5508 | A |
| 14 | chr14: 14840602 | 14840602 | C | 0.3767 | 0.295 | T | 0.6233 | 0.705 | T |
| 14 | chr14: 14840707 | 14840707 | C | 0.3767 | 0.295 | T | 0.6233 | 0.705 | T |
| 14 | chr14: 14866084 | 14866084 | G | 0.5233 | 0.44 | A | 0.4767 | 0.56 | A |
| 14 | chr14: 14869184 | 14869184 | A | 0.3567 | 0.2675 | G | 0.6433 | 0.7325 | G |
| 14 | chr14: 14923231 | 14923231 | A | 0.35 | 0.265 | G | 0.65 | 0.735 | G |
| 20 | chr20: 41512961 | 41512961 | C | 0.54 | 0.395 | A | 0.46 | 6.05E−01 | C |
| 20 | chr20: 41543010 | 41543010 | A | 0.604 | 0.5025 | G | 0.396 | 0.4975 | A |
| 20 | chr20: 41614101 | 41614101 | A | 0.6033 | 0.5025 | G | 0.3967 | 0.4975 | A |
| 20 | chr20: 41614453 | 41614453 | G | 0.8811 | 0.8495 | A | 0.1189 | 0.1505 | G |
| 20 | chr20: 41662902 | 41662902 | A | 0.6007 | 0.5026 | G | 0.3993 | 0.4974 | A |
| 20 | chr20: 41712898 | 41712898 | A | 0.6367 | 0.5125 | G | 0.3633 | 0.4875 | A |
| 20 | chr20: 41732334 | 41732334 | T | 0.6367 | 0.5125 | C | 0.3633 | 0.4875 | T |
| 20 | chr20: 41733976 | 41733976 | G | 0.6367 | 0.5125 | A | 0.3633 | 0.4875 | G |
| 20 | chr20: 41828740 | 41828740 | T | 0.527 | 0.3636 | C | 0.473 | 6.36E−01 | C |
| 20 | chr20: 41909338 | 41909338 | C | 0.6567 | 0.553 | T | 0.3433 | 0.447 | C |
| 20 | chr20: 41927603 | 41927603 | T | 0.5963 | 0.4286 | C | 0.4037 | 5.71E−01 | T |
| 20 | chr20: 41930509 | 41930509 | G | 0.59 | 0.4425 | A | 0.41 | 5.58E−01 | G |
| 20 | chr20: 41933198 | 41933198 | G | 0.59 | 0.4425 | A | 0.41 | 5.58E−01 | G |
| 20 | chr20: 41951828 | 41951828 | T | 0.59 | 0.4425 | C | 0.41 | 5.58E−01 | T |
| 20 | chr20: 41970787 | 41970787 | G | 0.66 | 0.55 | A | 0.34 | 0.45 | G |
| 20 | chr20: 41972158 | 41972158 | C | 0.7133 | 0.5975 | T | 0.2867 | 0.4025 | C |
| 20 | chr20: 41972956 | 41972956 | C | 0.5906 | 0.4422 | T | 0.4094 | 5.58E−01 | C |
| 20 | chr20: 41987996 | 41987996 | G | 0.59 | 0.4425 | A | 0.41 | 5.58E−01 | G |
| 20 | chr20: 41990290 | 41990290 | C | 0.59 | 0.4425 | T | 0.41 | 5.58E−01 | C |
| 20 | chr20: 41993220 | 41993220 | T | 0.59 | 0.4425 | G | 0.41 | 5.58E−01 | T |
| 20 | chr20: 42004062 | 42004062 | C | 0.6 | 0.495 | T | 0.4 | 0.505 | C |
| 20 | chr20: 42060186 | 42060186 | T | 0.5367 | 0.3675 | C | 0.4633 | 6.33E−01 | C |
| 20 | chr20: 42080147 | 42080147 | T | 0.3733 | 0.1175 | C | 0.6267 | 8.83E−01 | C |
| 20 | chr20: 42108401 | 42108401 | A | 0.66 | 0.54 | G | 0.34 | 0.46 | G |
| 20 | chr20: 42111613 | 42111613 | G | 0.6286 | 0.5281 | A | 0.3714 | 0.4719 | A |
| 20 | chr20: 42114307 | 42114307 | A | 0.66 | 0.54 | G | 0.34 | 0.46 | G |
| 20 | chr20: 42115073 | 42115073 | G | 0.6533 | 0.535 | A | 0.3467 | 0.465 | A |
| 20 | chr20: 42117345 | 42117345 | T | 0.66 | 0.54 | G | 0.34 | 0.46 | G |
| 20 | chr20: 42131456 | 42131456 | A | 0.5733 | 0.4 | G | 0.4267 | 6.00E−01 | G |
| 20 | chr20: 42131853 | 42131853 | G | 0.6367 | 0.5075 | A | 0.3633 | 4.93E−01 | A |
| 20 | chr20: 47886402 | 47886402 | C | 0.3567 | 0.24 | T | 0.6433 | 7.60E−01 | T |
| 20 | chr20: 47899650 | 47899650 | A | 0.3633 | 0.2375 | C | 0.6367 | 7.63E−01 | C |
| 20 | chr20: 48051957 | 48051957 | G | 0.4333 | 0.3492 | A | 0.5667 | 0.6508 | G |
| 20 | chr20: 48052681 | 48052681 | C | 0.36 | 0.2375 | T | 0.64 | 7.63E−01 | T |
| 20 | chr20: 48055355 | 48055355 | G | 0.4233 | 0.3425 | A | 0.5767 | 0.6575 | A |
| 20 | chr20: 48056097 | 48056097 | G | 0.1544 | 0.0804 | A | 0.8456 | 0.9196 | G |
| 20 | chr20: 48056581 | 48056581 | T | 0.4362 | 0.3475 | A | 0.5638 | 0.6525 | T |
| 20 | chr20: 48059078 | 48059078 | T | 0.36 | 0.235 | C | 0.64 | 7.65E−01 | C |
| 20 | chr20: 48060281 | 48060281 | G | 0.4362 | 0.3475 | A | 0.5638 | 0.6525 | G |
| 20 | chr20: 48062375 | 48062375 | C | 0.4333 | 0.3475 | T | 0.5667 | 0.6525 | C |
| 20 | chr20: 48062389 | 48062389 | G | 0.4262 | 0.345 | C | 0.5738 | 0.655 | G |
| 20 | chr20: 48062854 | 48062854 | G | 0.3667 | 0.2375 | A | 0.6333 | 7.63E−01 | G |
| 20 | chr20: 48072724 | 48072724 | A | 0.3867 | 0.2814 | G | 0.6133 | 0.7186 | G |
| 20 | chr20: 48111692 | 48111692 | T | 0.36 | 0.23 | C | 0.64 | 7.70E−01 | C |
| 20 | chr20: 48112205 | 48112205 | T | 0.36 | 0.2312 | C | 0.64 | 7.69E−01 | C |
| 20 | chr20: 48117256 | 48117256 | A | 0.36 | 0.2325 | G | 0.64 | 7.68E−01 | G |
| 20 | chr20: 48130277 | 48130277 | G | 0.43 | 0.3425 | A | 0.57 | 0.6575 | G |
| 20 | chr20: 48150406 | 48150406 | G | 0.3933 | 0.295 | A | 0.6067 | 0.705 | A |
| 20 | chr20: 48158297 | 48158297 | C | 0.3933 | 0.29 | G | 0.6067 | 0.71 | G |
| 20 | chr20: 45159029 | 48159029 | A | 0.3933 | 0.29 | G | 0.6067 | 0.71 | G |
| 20 | chr20: 48160311 | 48160311 | C | 0.42 | 0.3375 | G | 0.58 | 0.6625 | G |
| 20 | chr20: 48162500 | 48162500 | G | 0.3933 | 0.29 | A | 0.6067 | 0.71 | A |
| 20 | chr20: 48259767 | 48259767 | T | 0.4167 | 0.31 | C | 0.5833 | 0.69 | C |
| 20 | chr20: 48260231 | 48260231 | G | 0.4252 | 0.3141 | A | 0.5748 | 0.6859 | A |
| 20 | chr20: 48377580 | 48377580 | A | 0.3667 | 0.2375 | C | 0.6333 | 7.63E−01 | A |
| 20 | chr20: 48429591 | 48429591 | A | 0.3967 | 0.3065 | C | 0.6033 | 0.6935 | C |
| 20 | chr20: 48437593 | 48437593 | T | 0.4252 | 0.3434 | C | 0.5748 | 0.6566 | T |
| 20 | chr20: 48520099 | 48520099 | T | 0.3667 | 0.24 | C | 0.6333 | 7.60E−01 | C |
| 20 | chr20: 48599799 | 48599799 | A | 0.3667 | 0.2412 | C | 0.6333 | 7.59E−01 | C |
| 20 | chr20: 48601051 | 48601051 | C | 0.5 | 0.43 | T | 0.5 | 0.57 | C |
| 20 | chr20: 48650307 | 48650307 | A | 0.3931 | 0.3005 | G | 0.6069 | 0.6995 | A |
| 20 | chr20: 48704449 | 48704449 | C | 0.4567 | 0.37 | T | 0.3433 | 0.63 | T |
| 20 | chr20: 48743303 | 48743303 | G | 0.3267 | 0.2725 | A | 0.6733 | 0.7275 | G |
| 20 | chr20: 48743330 | 48743330 | T | 0.46 | 0.3725 | C | 0.54 | 0.6275 | T |
| 20 | chr20: 48744441 | 48744441 | G | 0.4567 | 0.3725 | A | 0.5433 | 0.6275 | G |
| 20 | chr20: 48756142 | 48756142 | G | 0.4267 | 0.3241 | T | 0.5733 | 0.6759 | T |
| 20 | chr20: 48756169 | 48756169 | C | 0.4333 | 0.3275 | T | 0.5667 | 0.6725 | C |
| 20 | chr20: 48802224 | 48802224 | A | 0.453 | 0.37 | G | 0.547 | 0.63 | A |
| 20 | chr20: 48804130 | 48804130 | G | 0.4633 | 0.3725 | A | 0.5367 | 0.6275 | G |
| 20 | chr20: 48811857 | 48811857 | A | 0.4567 | 0.365 | G | 0.5433 | 0.635 | A |
| 20 | chr20: 48841374 | 48841374 | G | 0.4067 | 0.295 | A | 0.5933 | 0.705 | G |
| 20 | chr20: 48855117 | 48855117 | A | 0.98333 | 0.955 | G | 0.01667 | 0.045 | G |
| 20 | chr20: 48906397 | 48906397 | T | 0.42 | 0.299 | C | 0.58 | 7.01E−01 | T |
| 20 | chr20: 49051904 | 49051904 | C | 0.3733 | 0.2775 | T | 0.6267 | 0.7225 | T |
| 20 | chr20: 49201505 | 49201505 | G | 0.36 | 0.225 | A | 0.64 | 7.75E−01 | A |
| 20 | chr20: 49479706 | 49479706 | A | 0.90667 | 0.87 | G | 0.09333 | 0.13 | A |
| 20 | chr20: 49671452 | 49671452 | G | 0.46 | 0.3925 | A | 0.54 | 0.6075 | G |
| 20 | chr20: 49687024 | 49687024 | G | 0.36 | 0.23 | A | 0.64 | 7.70E−01 | G |
| 20 | chr20: 49691940 | 49691940 | A | 0.3567 | 0.225 | G | 0.6433 | 7.75E−01 | A |

Ref = nucleotide identity in Boxer reference genome,
A1 = risk allele,
A2 = non-risk allele.

### Discussion

All hyaluronidase genes are positioned in two clusters in the dog genome, on chromosomes 14 and 20, where the two GWAS top loci are found. It is highly unlikely that both clusters should be identified in the genome-wide analyses by chance. Therefore, the hyaluronidase enzymes are potential candidates for involvement in the etiology of MCC risk in this breed. These findings suggest that the HA pathway is a major player in canine MCC predisposition. The biological function of hyaluronic acid depends on its molecular mass and low molecular weight HA promotes angiogenesis and signalling pathways involved in cancer progression [ref. 25,26]. The predisposing hyaluronidase mutations in the GR cohort could change the HA balance, which in turn would modify the extracellular environment of the cell to create a favourable tumour microenvironment.

In addition, the data herein show that a mutation in the GNAI2 gene introducing an alternative splice form of this gene is linked with the risk haplotype and is strongly associated with the disease. GNAI2 is a regulator of G-protein coupled receptors and also a negative regulator of intracellular cAMP. It therefore has an important role in cell signalling and proliferation and altered function of this gene can be oncogenic.

The findings from this GWAS study suggests a role for HA turnover in MCC in GRs. This study also demonstrates the benefits from mapping genetic risk factors underlying complex diseases within high-risk dog breeds with large effect sizes may be present. The results herein raise the potential that the hyaluronic acid metabolic pathway could also be a risk factor in human mastocytosis.

### Example 2

### Methods

To identify additional variants in the most associated regions, sequence capture library of the associated regions was performed on DNA from 8 American and 7 European individuals. The libraries were sequenced on Illumina HiSeq. New SNPs identified from the sequencing data, in the associated regions on chr 20 and chr 14, were evaluated in the full GWAS cohort and additional American cases and controls by Sequenome genotyping.

### Results

Additional SNPs identified and their associated p-values are listed in Table 8.

TABLE 8
Additional SNPs.

| | | | | Allele | Allele | | Allele | Allele | | |
|---|---|---|---|---|---|---|---|---|---|---|
| | | | | freq | freq | | freq | freq | | |
| CHR | SNP | POSITION | A1 | affected | control | A2 | affected | control | P-value | REF |
| 14 | chr14: 14653880 | 14653880 | C | 0.6111 | 0.4426 | T | 0.3889 | 0.5574 | 8.82E−04 | T |
| 14 | chr14: 14666424 | 14666424 | C | 0.7308 | 0.5244 | T | 0.2692 | 0.4756 | 3.73E−05 | T |
| 14 | chr14: 14682089 | 14682089 | T | 0.7812 | 0.5966 | C | 0.2188 | 0.4034 | 1.22E−04 | T |
| 14 | chr14: 14685602 | 14685602 | G | 0.8188 | 0.6458 | A | 0.1812 | 0.3542 | 1.75E−04 | G |
| 14 | chr14: 14685771 | 14685771 | G | 0.7938 | 0.6066 | T | 0.2062 | 0.3934 | 7.91E−05 | G |
| 20 | chr20: 41512961 | 41512961 | C | 0.5674 | 0.4148 | A | 0.4326 | 0.5852 | 1.19E−04 | C |
| 20 | chr20: 41543010 | 41543010 | A | 0.6403 | 0.5055 | G | 0.3597 | 0.4945 | 6.33E−04 | A |
| 20 | chr20: 41712898 | 41712898 | A | 0.6608 | 0.5134 | G | 0.3392 | 0.4866 | 1.48E−04 | A |
| 20 | chr20: 41732334 | 41732334 | T | 0.675 | 0.5108 | C | 0.325 | 0.4892 | 2.65E−05 | T |
| 20 | chr20: 41733976 | 41733976 | G | 0.6655 | 0.5189 | A | 0.3345 | 0.4811 | 1.65E−04 | G |
| 20 | chr20: 41828740 | 41828740 | T | 0.5468 | 0.3743 | C | 0.4532 | 0.6257 | 1.31E−05 | C |
| 20 | chr20: 41927603 | 41927603 | T | 0.6127 | 0.4383 | C | 0.3873 | 0.5617 | 1.11E−04 | T |
| 20 | chr20: 41933198 | 41933198 | G | 0.6119 | 0.457 | A | 0.3881 | 0.543 | 8.01E−05 | G |
| 20 | chr20: 41970787 | 41970787 | G | 0.6901 | 0.5568 | A | 0.3099 | 0.4432 | 5.13E−04 | G |
| 20 | chr20: 41972158 | 41972158 | C | 0.7359 | 0.6033 | T | 0.2641 | 0.3967 | 3.88E−04 | C |
| 20 | chr20: 41972956 | 41972956 | C | 0.6268 | 0.4574 | T | 0.3732 | 0.5426 | 1.59E−05 | C |
| 20 | chr20: 41987996 | 41987996 | G | 0.6232 | 0.4568 | A | 0.3768 | 0.5432 | 2.36E−05 | G |
| 20 | chr20: 41990290 | 41990290 | C | 0.6277 | 0.4617 | T | 0.3723 | 0.5383 | 2.70E−05 | C |
| 20 | chr20: 41993220 | 41993220 | T | 0.6181 | 0.4568 | G | 0.3819 | 0.5432 | 3.93E−05 | T |
| 20 | chr20: 42060186 | 42060186 | T | 0.5766 | 0.3846 | C | 0.4234 | 0.6154 | 1.49E−06 | C |
| 20 | chr20: 42080147 | 42080147 | T | 0.4028 | 0.1243 | C | 0.5972 | 0.8757 | 1.23E−16 | C |
| 20 | chr20: 42108401 | 42108401 | A | 0.6957 | 0.5405 | G | 0.3043 | 0.4595 | 6.54E−05 | G |
| 20 | chr20: 42114307 | 42114307 | A | 0.6972 | 0.5405 | G | 0.3028 | 0.4595 | 4.74E−05 | G |
| 20 | chr20: 42115073 | 42115073 | G | 0.6884 | 0.5351 | A | 0.3116 | 0.4649 | 8.33E−05 | A |
| 20 | chr20: 42117345 | 42117345 | T | 0.6879 | 0.5405 | G | 0.3121 | 0.4595 | 1.37E−04 | G |
| 20 | chr20: 42131456 | 42131456 | A | 0.6064 | 0.4127 | G | 0.3936 | 0.5873 | 8.52E−07 | G |
| 20 | chr20: 42131853 | 42131853 | G | 0.6655 | 0.5081 | A | 0.3345 | 0.4919 | 6.04E−05 | A |
| 20 | chr20: 47886402 | 47886402 | C | 0.3821 | 0.2297 | T | 0.6179 | 0.7703 | 2.47E−05 | T |
| 20 | chr20: 47899650 | 47899650 | A | 0.3811 | 0.2283 | C | 0.6189 | 0.7717 | 2.12E−05 | C |
| 20 | chr20: 48052681 | 48052681 | C | 0.3908 | 0.227 | T | 0.6092 | 0.773 | 5.65E−06 | T |
| 20 | chr20: 48056097 | 48056097 | G | 0.1884 | 0.07065 | A | 0.8116 | 0.92935 | 5.83E−06 | G |
| 20 | chr20: 48059078 | 48059078 | T | 0.3854 | 0.2302 | C | 0.6146 | 0.7698 | 1.41E−05 | C |
| 20 | chr20: 48062854 | 48062854 | G | 0.3881 | 0.2328 | A | 0.6119 | 0.7672 | 1.52E−05 | G |
| 20 | chr20: 48072724 | 48072724 | A | 0.4143 | 0.265 | G | 0.5857 | 0.735 | 6.36E−05 | G |
| 20 | chr20: 48111692 | 48111692 | T | 0.3873 | 0.2255 | C | 0.6127 | 0.7745 | 7.23E−06 | C |
| 20 | chr20: 48112205 | 48112205 | T | 0.3854 | 0.2283 | C | 0.6146 | 0.7717 | 1.24E−05 | C |
| 20 | chr20: 48117256 | 48117256 | A | 0.3723 | 0.2285 | G | 0.6277 | 0.7715 | 6.00E−05 | G |
| 20 | chr20: 48158297 | 48158297 | C | 0.4266 | 0.2962 | G | 0.5734 | 0.7038 | 5.39E−04 | G |
| 20 | chr20: 48159029 | 48159029 | A | 0.4414 | 0.2946 | G | 0.5586 | 0.7054 | 9.57E−05 | G |
| 20 | chr20: 48162500 | 48162500 | G | 0.4291 | 0.2946 | A | 0.5709 | 0.7054 | 3.70E−04 | A |
| 20 | chr20: 48259767 | 48259767 | T | 0.4371 | 0.3095 | C | 0.5629 | 0.6905 | 7.21E−04 | C |
| 20 | chr20: 48260231 | 48260231 | G | 0.4424 | 0.3155 | A | 0.5576 | 0.6845 | 8.98E−04 | A |
| 20 | chr20: 48377580 | 48377580 | A | 0.3944 | 0.2324 | C | 0.6056 | 0.7676 | 7.91E−06 | A |
| 20 | chr20: 48520099 | 48520099 | T | 0.3803 | 0.2366 | C | 0.6197 | 0.7634 | 6.76E−05 | C |
| 20 | chr20: 48756142 | 48756142 | G | 0.4784 | 0.3324 | T | 0.5216 | 0.6676 | 1.68E−04 | T |
| 20 | chr20: 48756169 | 48756169 | C | 0.4613 | 0.3306 | T | 0.5387 | 0.6694 | 6.66E−04 | C |
| 20 | chr20: 48841374 | 48841374 | G | 0.4321 | 0.2957 | A | 0.5679 | 0.7043 | 3.11E−04 | G |
| 20 | chr20: 48906397 | 48906397 | T | 0.4384 | 0.3033 | C | 0.5616 | 0.6967 | 4.18E−04 | T |
| 20 | chr20: 49051904 | 49051904 | C | 0.3944 | 0.2698 | T | 0.6056 | 0.7302 | 6.98E−04 | T |
| 20 | chr20: 49687024 | 49687024 | G | 0.3865 | 0.2324 | A | 0.6135 | 0.7676 | 2.07E−05 | G |
| 20 | chr20: 49691940 | 49691940 | A | 0.3671 | 0.2231 | G | 0.6329 | 0.7769 | 5.04E−05 | A |

### REFERENCES

1. Amon, U., Hartmann, K., Horny, H. P. & Nowak, A. Mastocytosis—an update. Journal der Deutschen Dermatologischen Gesellschaft=Journal of the German Society of Dermatology: JDDG 8, 695-711; quiz 712 (2010).
2. Laine, E., Chauvot de Beauchene, I., Perahia, D., Auclair, C. & Tchertanov, L. Mutation D816V alters the internal structure and dynamics of c-KIT receptor cytoplasmic region: implications for dimerization and activation mechanisms. PLoS computational biology 7, e1002068 (2011).
3. Bodemer, C. et al. Pediatric mastocytosis is a clonal disease associated with D816V and other activating c-KIT mutations. The Journal of investigative dermatology 130, 804-15 (2010).
4. Blackwood, L. et al. European consensus document on mast cell tumours in dogs and cats. Veterinary and comparative oncology 10, e1-e29 (2012).
5. Letard, S. et al. Gain-of-function mutations in the extracellular domain of KIT are common in canine mast cell tumors. Molecular cancer research: MCR 6, 1137-45 (2008).
6. Misdorp, W. Mast cells and canine mast cell tumours. A review. The Veterinary quarterly 26, 156-69 (2004).
7. Broesby-Olsen, S., Kristensen, T. K., Moller, M. B., Bindslev-Jensen, C. & Vestergaard, H. Adult-onset systemic mastocytosis in monozygotic twins with KIT D816V and JAK2 V617F mutations. The Journal of allergy and clinical immunology 130, 806-8 (2012).
8. Rosbotham, J. L. et al. Lack of c-kit mutation in familial urticaria pigmentosa. The British journal of dermatology 140, 849-52 (1999).
9. Miller, D. M. The occurrence of mast cell tumors in young Shar-Peis. Journal of veterinary diagnostic investigation: official publication of the American Association of Veterinary Laboratory Diagnosticians, Inc 7, 360-3 (1995).
10. White, C. R., Hohenhaus, A. E., Kelsey, J. & Procter-Gray, E. Cutaneous MCTs: associations with spay/neuter status, breed, body size, and phylogenetic cluster. Journal of the American Animal Hospital Association 47, 210-6 (2011).
11. Seguin, B. et al. Recurrence rate, clinical outcome, and cellular proliferation indices as prognostic indicators after incomplete surgical excision of cutaneous grade II mast cell tumors: 28 dogs (1994-2002). Journal of veterinary internal medicine/American College of Veterinary Internal Medicine 20, 933-40 (2006).
12. Lindblad-Toh, K. et al. Genome sequence, comparative analysis and haplotype structure of the domestic dog. Nature 438, 803-19 (2005).
13. Karlsson, E. K. et al. Efficient mapping of mendelian traits in dogs through genome-wide association. Nat Genet 39, 1321-8 (2007).
14. Ji, L., Minna, J. D. & Roth, J. A. 3p21.3 tumor suppressor cluster: prospects for translational applications. Future oncology 1, 79-92 (2005).
15. Hesson, L. B., Cooper, W. N. & Latif, F. Evaluation of the 3p21.3 tumour-suppressor gene cluster. Oncogene 26, 7283-301 (2007).
16. Olsson, M. et al. A Novel Unstable Duplication Upstream of HAS2 Predisposes to a Breed-Defining Skin Phenotype and a Periodic Fever Syndrome in Chinese Shar-Pei Dogs. PLoS Genet 7, e1001332.
17. Bouga, H. et al. Involvement of hyaluronidases in colorectal cancer. BMC cancer 10, 499 (2010).
18. Paiva, P. et al. Expression patterns of hyaluronan, hyaluronan synthases and hyaluronidases indicate a role for hyaluronan in the progression of endometrial cancer. Gynecologic oncology 98, 193-202 (2005).
19. Bertrand, P. et al. Expression of HYAL2 mRNA, hyaluronan and hyaluronidase in B-cell non-Hodgkin lymphoma: relationship with tumor aggressiveness. International journal of cancer. Journal international du cancer 113, 207-12 (2005).
20. Kramer, M. W. et al. Association of hyaluronic acid family members (HAS1, HAS2, and HYAL-1) with bladder cancer diagnosis and prognosis. Cancer 117, 1197-209 (2011).
21. Liu, D. et al. Expression of hyaluronidase by tumor cells induces angiogenesis in vivo. Proceedings of the National Academy of Sciences of the United States of America 93, 7832-7 (1996).
22. Itano, N., Zhuo, L. & Kimata, K. Impact of the hyaluronan-rich tumor microenvironment on cancer initiation and progression. Cancer science 99, 1720-5 (2008).
23. Corte, M. D. et al. Analysis of the expression of hyaluronan in intraductal and invasive carcinomas of the breast. Journal of cancer research and clinical oncology 136, 745-50 (2010).
24. Tammi, R. H. et al. Hyaluronan in human tumors: pathobiological and prognostic messages from cell-associated and stromal hyaluronan. Seminars in cancer biology 18, 288-95 (2008).
25. Girish, K. S. & Kemparaju, K. The magic glue hyaluronan and its eraser hyaluronidase: a biological overview. Life sciences 80, 1921-43 (2007).
26. Stern, R., Asari, A. A. & Sugahara, K. N. Hyaluronan fragments: an information-rich system. European journal of cell biology 85, 699-715 (2006).
27. Takano, H. et al. Restriction of mast cell proliferation through hyaluronan synthesis by co-cultured fibroblasts. Biological & pharmaceutical bulletin 35, 408-12 (2012).
28. Guo, N., Baglole, C. J., O'Loughlin, C. W., Feldon, S. E. & Phipps, R. P. Mast cell-derived prostaglandin D2 controls hyaluronan synthesis in human orbital fibroblasts via DP1 activation: implications for thyroid eye disease. The Journal of biological chemistry 285, 15794-804 (2010).
29. Nagata, Y. et al. Secretion of hyaluronic acid from synovial fibroblasts is enhanced by histamine: a newly observed metabolic effect of histamine. The Journal of laboratory and clinical medicine 120, 707-12 (1992).
30. Nilsson, G. & Nilsson, K. Effects of interleukin (IL)-13 on immediate-early response gene expression, phenotype and differentiation of human mast cells. Comparison with IL-4. European journal of immunology 25, 870-3 (1995).
31. Mani, S. A. et al. The epithelial-mesenchymal transition generates cells with properties of stem cells. Cell 133, 704-15 (2008).
32. Zoller, M. CD44: can a cancer-initiating cell profit from an abundantly expressed molecule? Nature reviews. Cancer 11, 254-67 (2011).
33. Garcia-Closas, M. et al. Collection of genomic DNA from adults in epidemiological studies by buccal cytobrush and mouthwash. Cancer epidemiology, biomarkers & prevention: a publication of the American Association for Cancer Research, cosponsored by the American Society of Preventive Oncology 10, 687-96 (2001).
34. Miller, S. A., Dykes, D. D. & Polesky, H. A simple salting out procedure for extracting DNA from human nucleated cells. Nucleic acids research 16, 1215 (1988).
35. Vaysse, A. et al. Identification of genomic regions associated with phenotypic variation between dog breeds using selection mapping. PLoS genetics 7, e1002316 (2011).
36. Purcell, S. et al. PLINK: a tool set for whole-genome association and population-based linkage analyses. Am J Hum Genet 81, 559-75 (2007).
37. Yang, J., Lee, S. H., Goddard, M. E. & Visscher, P. M. GCTA: a tool for genome-wide complex trait analysis. American journal of human genetics 88, 76-82 (2011).
38. Team, R. D. C. R: A language and environment for statistical computing. (R Foundation for Statistical Computing, Vienna, Austria, 2008).
39. Aulchenko, Y. S., Ripke, S., Isaacs, A. & van Duijn, C. M. GenABEL: an R library for genome-wide association analysis. Bioinformatics 23, 1294-6 (2007).
40. Barrett, J. C., Fry, B., Maller, J. & Daly, M. J. Haploview: analysis and visualization of LD and haplotype maps. Bioinformatics 21, 263-5 (2005).

Without further elaboration, it is believed that one skilled in the art can, based on the above description, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

The indefinite articles “a” and “an,” as used herein in the specification and in the claims, unless clearly indicated to the contrary, should be understood to mean “at least one.”

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method, comprising:
(a) analyzing genomic DNA from a canine subject for the presence of a single nucleotide polymorphism (SNP) selected from:
i) one or more chromosome 5 SNPs,
ii) a chromosome 8 SNP TIGRP2P118921,
iii) one or more chromosome 14 SNPs, and
iv) one or more chromosome 20 SNPs; and
(b) identifying a canine subject having the SNP as a subject at elevated risk of developing a mast cell cancer or having an undiagnosed mast cell cancer.

2. The method of claim 1, wherein the SNP is selected from:
one or more chromosome 14 SNPs, and
one or more chromosome 20 SNPs.

3. The method of claim 1 or 2, wherein the SNP is selected from one or more chromosome 14 SNPs.

4. The method of claim 3, wherein the SNP is selected from one or more chromosome 14 SNPs BICF2G630521558, BICF2G630521606, BICF2G630521619, BICF2G630521572, and BICF2P867665.

5. The method of claim 4, wherein the SNP is BICF2P867665.

6. The method of claim 1 or 2, wherein the wherein the SNP is selected from one or more chromosome 20 SNPs.

7. The method of claim 6, wherein the SNP is selected from one or more chromosome 20 SNPs BICF2S22934685, BICF2P1444805, BICF2P299292, BICF2P301921, and BICF2P623297.

8. The method of claim 7, wherein the SNP is BICF2P301921.

9. The method of claim 6, wherein the SNP is selected from one or more chromosome 20 SNPs BICF2P304809, BICF2P1310301, BICF2P1310305, BICF2P1231294, and BICF2P1185290.

10. The method of claim 9, wherein the SNP is BICF2P1185290.

11. The method of any one of claims 1 to 10, wherein the genomic DNA is obtained from a bodily fluid or tissue sample of the subject.

12. The method of 11, wherein the genomic DNA is obtained from a blood or saliva sample of the subject.

13. The method of any one of claims 1 to 12, wherein the genomic DNA is analyzed using a single nucleotide polymorphism (SNP) array.

14. The method of any one of claims 1 to 12, wherein the genomic DNA is analyzed using a bead array.

15. The method of any one of claims 1 to 12, wherein the genomic DNA is analyzed using a nucleic acid sequencing assay.

16. The method of claim 1, wherein the SNP is two or more SNPs.

17. The method of claim 1, wherein the SNP is three or more SNPs.

18. A method, comprising:
(a) analyzing genomic DNA from a canine subject for the presence of a risk haplotype selected from:
(i) a risk haplotype having chromosome coordinates Chr5:8.42-10.73 Mb,
(ii) a risk haplotype having chromosome coordinates Chr14:14.64-14.76 Mb,
(iii) a risk haplotype having chromosome coordinates Chr20:41.51-42.12 Mb,
(iv) a risk haplotype having chromosome coordinates Chr20:41.70-42.59 Mb, and
(v) a risk haplotype having chromosome coordinates Chr20:47.06-49.70 Mb; and
(b) identifying a canine subject having the risk haplotype as a subject at elevated risk of developing a mast cell cancer or having an undiagnosed mast cell cancer.

19. The method of claim 18, wherein the presence of the risk haplotype is detected by analyzing the genomic DNA for the presence of a SNP is selected from:
(a) Chr5:8.42-10.73 Mb SNPs BICF2P807873, BICF2P778319, BICF2P547394, BICF2P1347656, BICF2S2331073, BICF2S23025903, and BICF2S23519930,
(b) Chr14:14.64-14.76 Mb SNPs BICF2G630521558, BICF2G630521572, BICF2G630521606, BICF2G630521619, BICF2P867665, TIGRP2P186605, BICF2G630521678, BICF2G630521681, and BICF2G630521696,
(c) Chr20:41.51-42.12 Mb SNPs BICF2P453555, BICF2P372450, BICF2P271393, BICF2S22934685, BICF2S2295117,
(d) Chr20:41.70-42.59 Mb SNPs BICF2P453555, BICF2P372450, BICF2P271393, BICF2S22934685, BICF2S2295117, and
(e) Chr20:47.06-49.70 Mb SNPs BICF2P327134, BICF2P854185, BICF2P304809, BICF2P1310301, BICF2P1310305, BICF2P1231294, BICF2P541405, BICF2P112281, BICF2P1185290, and BICF2P1241961.

20. The method of claim 18 or 19, wherein the risk haplotype is selected from
the risk haplotype having chromosome coordinates Chr14:14.64-14.76 Mb,
the risk haplotype having chromosome coordinates Chr20:41.51-42.12 Mb,
the risk haplotype having chromosome coordinates Chr20:41.70-42.59 Mb, and
the risk haplotype having chromosome coordinates Chr20:47.06-49.70 Mb.

21. The method of any one of claims 18 to 20, wherein the risk haplotype is the risk haplotype having chromosome coordinates Chr14:14.64-14.76 Mb.

22. The method of any one of claims 18 to 20, wherein the risk haplotype is the risk haplotype having chromosome coordinates Chr20:41.51-42.12 Mb.

23. The method of any one of claims 18 to 20, wherein the risk haplotype is the risk haplotype having chromosome coordinates Chr20:41.70-42.59 Mb or the risk haplotype having chromosome coordinates Chr20:47.06-49.70 Mb.

24. The method of claim 23, wherein the risk haplotype is the risk haplotype having chromosome coordinates Chr20:47.06-49.70 Mb

25. The method of any one of claims 18 to 24, wherein the genomic DNA is obtained from a bodily fluid or tissue sample of the subject.

26. The method of claim 25, wherein the genomic DNA is obtained from a blood or saliva sample of the subject.

27. The method of any one of claims 18 to 26, wherein the genomic DNA is analyzed using a single nucleotide polymorphism (SNP) array.

28. The method of any one of claims 18 to 27, wherein the genomic DNA is analyzed using a bead array.

29. The method of any one of claims 18 to 27, wherein the genomic DNA is analyzed using a nucleic acid sequencing assay.

30. The method of claim 18, wherein the SNP is two or more SNPs.

31. The method of claim 18, wherein the SNP is three or more SNPs.

32. The method of claim 19, wherein the SNP is a group of SNPs selected from (a) to (e):
(a) Chr5:8.42-10.73 Mb SNPs BICF2P807873, BICF2P778319, BICF2P547394, BICF2P1347656, BICF2S2331073, BICF2S23025903, and BICF2S23519930,
(b) Chr14:14.64-14.76 Mb SNPs BICF2G630521558, BICF2G630521572, BICF2G630521606, BICF2G630521619, BICF2P867665, TIGRP2P186605, BICF2G630521678, BICF2G630521681, and BICF2G630521696,
(c) Chr20:41.51-42.12 Mb SNPs BICF2P453555, BICF2P372450, BICF2P271393, BICF2S22934685, BICF2S2295117,
(d) Chr20:41.70-42.59 Mb SNPs BICF2P453555, BICF2P372450, BICF2P271393, BICF2S22934685, BICF2S2295117, and
(e) Chr20:47.06-49.70 Mb SNPs BICF2P327134, BICF2P854185, BICF2P304809, BICF2P1310301, BICF2P1310305, BICF2P1231294, BICF2P541405, BICF2P112281, BICF2P1185290, and BICF2P1241961.

33. The method of claim 18, wherein the risk haplotype is two or more risk haplotypes.

34. The method of claim 18, wherein the risk haplotype is three or more risk haplotypes.

35. A method, comprising:
(a) analyzing genomic DNA from a canine subject for the presence of a mutation in a gene selected from:
(i) one or more genes located within a risk haplotype having chromosome coordinates Chr5:8.42-10.73 Mb,
(ii) one or more genes within 500 Kb of TIGRP2P118921 on chromosome 8,
(iii) one or more genes located within a risk haplotype having chromosome coordinates Chr14:14.64-14.76 Mb,
(iv) one or more genes located within a risk haplotype having chromosome coordinates Chr20:41.51-42.12 Mb,
(v) one or more genes located within a risk haplotype having chromosome coordinates Chr20:41.70-42.59 Mb, and
(vi) one or more genes located within a risk haplotype having chromosome coordinates Chr20:47.06-49.70 Mb, and
(b) identifying a canine subject having the mutation as a subject at elevated risk of developing a mast cell cancer or having an undiagnosed mast cell cancer.

36. The method of claim 35, wherein the gene is selected from one or more genes located within a risk haplotype having chromosome coordinates Chr14:14.64-14.76 Mb.

37. The method of claim 36, wherein the gene is selected from SPAM1, HYAL4, and HYALP1.

38. The method of claim 35, wherein the gene is selected from one or more genes located within a risk haplotype having chromosome coordinates Chr20:41.51-42.12 Mb or one or more genes located within a risk haplotype having chromosome coordinates Chr20:47.06-49.70 Mb.

39. The method of claim 35, wherein the gene is selected from one or more genes located within a risk haplotype having chromosome coordinates Chr20:47.06-49.70 Mb.

40. The method of claim 35, wherein the gene is selected from one or more genes located within a risk haplotype having chromosome coordinates Chr20:41.51-42.12 Mb.

41. The method of claim 40, wherein the gene is selected from DOCK3, ENSCAFG00000010275, MAPKAPK3, CISH, HEMK1, C3orf18, CACNA2D2, TMEM115, NPRL2, ZMYND10, RASSF1, TUSC2, HYAL2, HYAL1, HYAL3, C3orf45, ENSCAFG00000010719, GNAI2_CANFA, and ENSCAFG00000010754.

42. The method of claim 35, wherein the gene is selected from MAPKAPK3, CISH, HEMK1, C3orf18, CACNA2D2, TMEM115, CYB561D2, NPRL2, ZMYND10, RASSF1, TUSC2, HYAL2, HYAL1, HYAL3, C3oef45, GNAI2, ENSCAFG00000010719, and ENSCAFG00000010754.

43. The method of claim 42, wherein the gene is GNAI2.

44. The method of claim 35, wherein the gene is selected from HYAL1, HYAL2, HYAL3, SPAM1, HYAL4, and HYALP1.

45. The method of any one of claims 35 to 44, wherein the genomic DNA is obtained from a bodily fluid or tissue sample of the subject.

46. The method of claim 45, wherein the genomic DNA is obtained from a blood or saliva sample of the subject.

47. The method of any one of claims 35 to 46, wherein the genomic DNA is analyzed using a single nucleotide polymorphism (SNP) array.

48. The method of any one of claims 35 to 47, wherein the genomic DNA is analyzed using a bead array.

49. The method of any one of claims 35 to 47, wherein the genomic DNA is analyzed using a nucleic acid sequencing assay.

50. The method of claim 35, wherein the mutation is two or more mutations.

51. The method of claim 35, wherein the mutation is three or more mutations.

52. The method of claim 35, wherein the gene is two or more genes.

53. The method of claim 35, wherein the gene is three or more genes.

54. The method of any of the foregoing claims, wherein the mast cell cancer is a mast cell cancer located in the skin of the subject.

55. The method of any of the foregoing claims, wherein the canine subject is a descendent of a Golden Retriever.

56. The method of any of the foregoing claims, wherein the canine subject is a Golden Retriever.

57. A method, comprising:
(a) analyzing genomic DNA in a sample from a subject for presence of a mutation in a gene selected from
(i) one or more genes located within a risk haplotype having chromosome coordinates Chr5:8.42-10.73 Mb, or an orthologue of such a gene,
(ii) one or more genes within 500 Kb of TIGRP2P118921 on chromosome 8,
(iii) one or more genes located within a risk haplotype having chromosome coordinates Chr14:14.64-14.76 Mb, or an orthologue of such a gene,
(iv) one or more genes located within a risk haplotype having chromosome coordinates Chr20:41.51-42.12 Mb, or an orthologue of such a gene,
(v) one or more genes located within a risk haplotype having chromosome coordinates Chr20:41.70-42.59 Mb, or an orthologue of such a gene, and
(vi) one or more genes located within a risk haplotype having chromosome coordinates Chr20:47.06-49.70 Mb or an orthologue of such a gene; and
(b) identifying a subject having the mutation as a subject at elevated risk of developing a mast cell cancer or having an undiagnosed mast cell cancer.

58. The method of claim 57, wherein the subject is a human subject.

59. The method of claim 57, wherein the subject is a canine subject.

60. The method of any one of claims 57 to 59, wherein the genomic DNA is obtained from a bodily fluid or tissue sample of the subject.

61. The method of claim 60, wherein the genomic DNA is obtained from a blood or saliva sample of the subject.

62. The method of any one of claims 57 to 61, wherein the genomic DNA is analyzed using a single nucleotide polymorphism (SNP) array.

63. The method of any one of claims 57 to 63, wherein the genomic DNA is analyzed using a bead array.

64. The method of any one of claims 57 to 63, wherein the genomic DNA is analyzed using a nucleic acid sequencing assay.

65. The method of any one of claims 57 to 64, wherein the mast cell cancer is a mast cell cancer located in the skin of the subject.

66. The method of claim 57, wherein the gene is two or more genes.

67. The method of claim 57, wherein the gene is three or more genes.

68. The method of claim 57, wherein the mutation is two or more mutations.

69. The method of claim 57, wherein the mutation is three or more mutations.
